(19) United States
(12) Patent Application Publication
(10) Pub. No.: US 2025/0268898 A1
(43) Pub. Date: Aug. 28, 2025

(54) METHODS OF TREATING SYNOVIAL SARCOMA PATHOGENESIS BY SUMO2 INHIBITION

(71) Applicant: Sanford Burnham Prebys Medical Discovery Institute, La Jolla, CA (US)

(72) Inventors: Aniruddha Jayant DESHPANDE, San Diego, CA (US); Rema IYER, San Diego, CA (US)

(21) Appl. No.: 19/037,764

(22) Filed: Jan. 27, 2025

Related U.S. Application Data
(60) Provisional application No. 63/574,638, filed on Apr. 4, 2024.
(60) Provisional application No. 63/624,924, filed on Jan. 25, 2024.

(51) Int. Cl. A61K 31/506; A61P 35/00

(52) U.S. Cl. CPC A61K 31/506; A61P 35/00

(57) ABSTRACT

Provided herein are methods and compositions for treating cancer. In some cases, the cancer comprises soft tissue sarcoma, such as synovial sarcoma. In some cases, the treatment comprises administering a therapeutically effective amount of an SS18-SSX fusion protein inhibitor to the subject. In some cases, the treatment comprises administering a therapeutically effective amount of SUMO2 inhibitor to the subject. The subject can be a pediatric subject or an adult subject.

### CROSS-REFERENCE

This application claims the benefit of U.S. Provisional Patent Application No. 63/624,924 filed Jan. 25, 2024, and U.S. Provisional Patent Application No. 63/574,638 filed Apr. 4, 2024. The contents of the above-referenced applications are incorporated by reference in their entirety.

### SEQUENCE LISTING

The instant application contains a Sequence Listing which has been filed electronically in XML format and is hereby incorporated by reference in its entirety. Said XML copy, created on Mar. 19, 2024, is named 42256-627.201_SL. XML and is 6,544 bytes in size.

### BACKGROUND

Synovial Sarcoma (SySa) is a soft tissue sarcoma that accounts for approximately 5% to 10% of all soft tissue sarcomas. SySa disproportionately affects children, adolescents, and young adults, often occurring in the extremities. Current treatment involves radiation and radical surgery including limb amputation, which are not universally effective in all patients. Therefore, there exists a need in the art for targeted therapies to treat SySa in children and adults. Tailoring therapies to treat SySa is a challenging undertaking due to the rarity of the disease and the resulting limited understanding of the underlying mechanisms for its pathogenesis.

### SUMMARY

Described herein are methods for treating a subject having Synovial Sarcoma (SySa) with a therapeutically effective amount of a small molecule.

In some embodiments, described herein is a method of reducing proliferation in a population of synovial sarcoma cells comprising treating a population with a compound inhibiting the conjugation of a small ubiquitin-like modifier 2 (SUMO2).

In some aspects, a compound described herein comprises a SUMO2-activating enzyme (SAE) inhibitor.

In some cases, an SAE described herein comprises a SUMO2-activating enzyme 1 (SAE1) or a SUMO2-activating enzyme 2 (SAE2).

In some aspects, a compound described herein comprises a compound or pharmaceutically acceptable salt thereof having the structure:

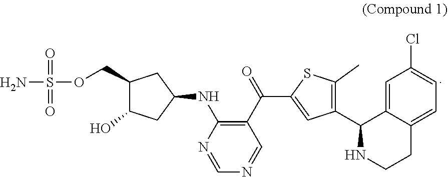 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

In some cases, a method described herein induces apoptosis or necrosis, or both, in a population of SySa cells.

In some embodiments, described herein is a method of reducing proliferation and inducing apoptosis in a population of cancer cells comprising treating the population with a compound that inhibits expression of an SS18-SSX fusion protein in the population of cells.

In some aspects, the SSX portion of a fusion protein described herein comprises SSX1, SSX2, or SSX4.

In some cases, a compound described herein comprises a small ubiquitin-like modifier 2 (SUMO-2) enzyme inhibitor.

In some aspects, a compound described herein inhibits SUMO2-activating enzyme 1 (SAE1) or SUMO2-activating enzyme 2 (SAE2).

In some cases, a population of cancer cells described herein comprises a population of SySa cells.

In some aspects, a method described herein induces a downregulation of one or more genes in a population of cancer cells.

In some cases, one or more genes described herein comprise genes selected from the group comprising CDX2, CBX5, SUZ12, CENPA, HOXA10, CENPF, TYMS, AURKB, UBE2C, or BCL2L11.

In some aspects, a method described herein induces an upregulation of one or more genes in a population of cancer cells.

In some cases, one or more genes described herein comprise genes selected from the group comprising GADD45B, KLF4, NCOA3, or BCL2L11.

In some aspects, a method described herein decreases the proliferation of a population of SySa cells compared to an otherwise identical population of non-synovial sarcoma cells.

In some embodiments, described herein is a method of reducing an expression of an SS18-SSX fusion protein in a population of cancer cells in a subject in need thereof, wherein the method comprises administering to the subject a small ubiquitin-like modifier 2 (SUMO-2) enzyme inhibitor.

In some cases, a method disclosed herein decreases the proliferation, increases apoptosis, or both, of a population of SySa cells compared to an otherwise identical population of non-synovial sarcoma cells.

In some embodiments, described herein is a method of reducing an expression of a SS18-SSX fusion protein in a population of cancer cells in a subject in need thereof, wherein the method comprises administering to the subject a compound or pharmaceutically acceptable salt thereof having the structure:

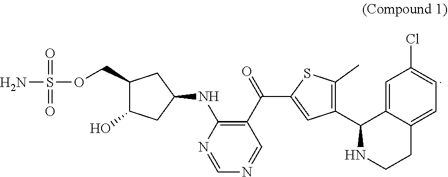 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

In some aspects, an SS18-SSX fusion protein disclosed herein comprises an SS18 domain and an SSX domain.

In some cases, the N-terminus of an SS18 domain disclosed herein is linked to the C-terminus of an SSX domain disclosed herein.

In some aspects, an SS18 domain disclosed herein comprises SEQ ID NO:1.

In some cases, an SSX domain disclosed herein is selected from the group consisting of an SSX1 domain, an SSX2 domain, an SSX3 domain, and an SSX4 domain.

In some aspects, an SSX1 domain disclosed herein comprises SEQ ID NO:2, the SSX2 domain comprises SEQ ID NO:3; the SSX3 domain comprises SEQ ID NO:4; or the SSX4 domain comprises SEQ ID NO:5.

In some cases, a method disclosed herein comprising administering to a subject in need thereof a composition comprising a compound or pharmaceutically acceptable salt of a compound disclosed herein.

In some aspects, a dosage of a compound disclosed herein is about 0.01 mg, about 0.5 mg, about 1 mg, about 2 mg, about 5 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, or more than about 150 mg, or an amount within a range defined by any of the preceding values.

In some cases, a composition disclosed herein is administered to an adult subject.

In some aspects, a composition disclosed herein is administered to a pediatric subject.

In yet another embodiment, disclosed herein is a method of reducing an expression of H2AK119Ub in a population of cancer cells in a subject, wherein the method comprises administering to the subject a small ubiquitin-like modifier 2 (SUMO-2) enzyme inhibitor.

In some cases, the compound inhibits SUMO2-activating enzyme 1 (SAE1) or SUMO2-activating enzyme 2 (SAE2). In some cases, the population of cancer cells comprises a population of synovial sarcoma cells. In some embodiments, the synovial sarcoma cells comprise a population of cell line 1273/99 cells. In other embodiments, the population of synovial sarcoma cells comprise a population of YAMATO-SS cells.

In some embodiments, the method further comprises inducing a downregulation of one or more genes in the population of cancer cells. In some cases, the SUMO-2 enzyme inhibitor is Compound 1, or pharmaceutically acceptable salt thereof, having the structure:

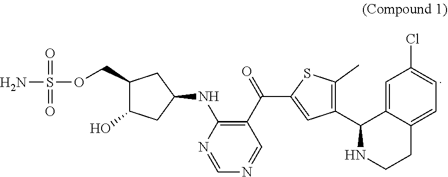 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

In some embodiments, the method further comprises measuring H2AK119Ub concentration or presence. In some cases, the measurement of H2AK119Ub concentration or presence is used to determine the level of SS18-SSX fusion protein downregulation. In some cases, the measurement of H2AK119Ub concentration or presence is used to determine the effectiveness of Compound 1.

In some embodiments, the method reduces Ki-67 expression in synovial sarcoma tumors by at least about 60% compared to a vehicle control. In some embodiments, the one or more downregulated genes are selected from a group of genes further comprising HOXC10, SMC2, or any combination thereof. In some embodiments, the one or more upregulated genes are selected from a group of genes further comprising CXCR4, GDF15, or combinations thereof.

In some embodiments, the population of synovial sarcoma cells comprises a population of SYO1, HS-SY-II, or ASKA-SS cells.

In some embodiments, the method further the method further comprises measuring H2AK119Ub chromatin occupancy at one or more synovial sarcoma direct target genes. In some embodiments, the one or more synovial sarcoma direct target genes comprises: CDX2, CBX5, SUZ12, CENPA, HOXA10, CENPF, TYMS, AURKB, UBE2C, BCL2L11, HOXC10, SMC2, GADD45B, KLF4, NCOA3, CXCR4, GDF15, or combinations thereof.

In some embodiments, the method further comprises measuring SS18-SSX concentration. In some embodiments, the measurement of SS18-SSX concentration is used to determine the level of SUMO-2 downregulation. In some embodiments, the measurement of SS18-SSX concentration is used to determine the effectiveness of Compound 1.

In some embodiments, the method further comprises measuring SS18-SSX chromatin occupancy at one or more synovial sarcoma direct target genes. In some embodiments, the one or more synovial sarcoma direct target genes comprises: CDX2, CBX5, SUZ12, CENPA, HOXA10, CENPF, TYMS, AURKB, UBE2C, BCL2L11, HOXC10, SMC2, GADD45B, KLF4, NCOA3, CXCR4, GDF15, or combinations thereof. In some embodiments, the measurement of SS18-SSX chromatin occupancy at synovial sarcoma direct target genes is used to determine the level of SUMO-2 downregulation. In some embodiments, the measurement of SS18-SSX chromatin occupancy at synovial sarcoma direct target genes is used to determine the effectiveness of Compound 1

In some embodiments, the measurement of H2AK119Ub chromatin occupancy at SySa direct target genes is used to determine the level of SS18-SSX fusion protein downregulation.

In some embodiments, wherein the measurement of H2AK119Ub chromatin occupancy at SySa direct target genes is used to determine the effectiveness of Compound 1. In some embodiments, the method further results in a 1.53-fold reduction of H2AK119Ub chromatin occupancy at one or more synovial sarcoma direct target genes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is a schematic depicting the complexing of the SS18-SSX fusion gene. FIG. 1B represents the synovial sarcoma cell lines of interest. FIG. 1C represents genes involved in synovial sarcoma. FIG. 1D represents a string analysis of complexes involved in synovial sarcoma. FIG. 1E is a heatmap representing quantified gene dependencies of non-SySa cell lines (left) with SySa cell lines (right). FIG. 1F is a scatter plot showing the relationship between gene dependency and differential transcript expression (log2 fold change FC differential expression) for all genes, with key synovial sarcoma selective essential genes labeled.

FIG. 2A depicts a CRISPR genetic target screening method using a synovial sarcoma cell line in vitro and in vivo in murine models. FIG. 2B depicts Robust Rank Aggregation (RRA) measurements for various genes of interest in vitro. FIG. 2C depicts Robust Rank Aggregation (RRA) measurements for various genes of interest in vivo. FIG. 2D depicts a representation of the number of genes of interest overlapping in vivo, in vitro, and in the Core SySa Oncogenic Program. FIG. 2E depicts data concerning the Chronos score of a gene of interest in synovial sarcoma cell lines in comparison to other cancer cell lines. FIG. 2F depicts pathway enrichment scores in the in vitro screens. −Log 10 False Discovery Rate (FDR) is shown on the X-axis. The size of the bubbles indicates normalized enrichment scores of each pathway. FIG. 2G depicts pathway enrichment scores in the in vivo screens. −Log 10 False Discovery Rate (FDR) is shown on the X-axis. The size of the bubbles indicates normalized enrichment scores of each pathway.

FIG. 3A depicts the structure of Compound 1. FIG. 3B depicts a function of Compound 1 in interrupting the SUMO cycle. FIGS. 3C-3E depict data measuring Relative Light Units (RLU) from the Cell Titer Glo Assay for various concentrations of Compound 1 in contact with synovial sarcoma cell types. FIG. 3F further depicts the viability of various SySa cell lines measured using Cell-Titer-Glo after 48 hrs. of treatment with varying concentrations of Compound 1. FIG. 3G depicts data measuring the percentage of Annexin in positive cells of different synovial sarcoma cell types in DMSO and in contact with Compound 1. FIG. 3H depicts flow cytometry data measuring percent Propidium Iodide (PI)-positive HS-SY-II (left) or SYO1 (right) cells treated with Compound 1. FIG. 3I depicts a heatmap quantifying viability of Aska-SS and SYO1 cell lines when administered Compound 1 at various dosages. Cell viability was affected in a Compound 1 concentration-dependent manner. FIG. 3J depicts crystal violet staining of colony forming-assays involving SYO1, HS-SY-II and 12273/99 cell lines treated with various concentrations of Compound 1.

FIG. 4A depicts data representing total RNA sequencing in units of −log of BH adjusted p-value for various log fold change values of various genes of interest. FIG. 4B depicts data representing total RNA sequencing in units of −log 10 adjusted p-value for various log fold change values of various genes of interest in SYO1 cells. FIG. 4C data representing total RNA sequencing in units of −log 10 adjusted p-value for various log fold change values of various genes of interest in SYO1 cells. FIG. 4D depicts a representation of FDR, size of the gene-set, and Normalized Enrichment Score (NES) for synovial sarcoma-involved genes of interest upregulated in DMSO and upregulated when contacted with Compound 1. FIG. 4E depicts data representing the enrichment score (ES) of target genes of interest upregulated or downregulated when in contact with Compound 1 as compared to DMSO. FIG. 4F depicts a heat map of upregulated and downregulated synovial sarcoma-involved target genes of interest in DMSO and contacting Compound 1. FIG. 4G depicts a heat map of target genes of interest in DMSO and contacting Compound 1. FIG. 4H depicts data measuring the enrichment score and ranked list metric for downregulation of genes associated with resistance to the commonly used anti-cancer agent Doxorubicin in contact with various concentrations of Compound 1. FIG. 4I depicts a heat map of the expression of target genes of interest in DMSO or Compound 1 treated HS-SY-II SySa cells.

FIG. 5A depicts data measuring the RNA log fold change for synovial sarcoma HS-SY-II cells contacted with various inducible shRNAs. FIG. 5B depicts a Western blot result example for HS-SY-II cells probed with various shRNAs specific to SUMO2. FIG. 5C depicts a Western blot result example for HS-SY-II cells probed with various shRNAs specific to SS18-SSX fusion. FIG. 5D depicts a Western blot result example for HS-SY-II cells probed with Compound 1. FIG. 5E depicts a Western blot result example for SYO1 cells probed with Compound 1.

FIG. 6A depicts a Western blot result example for SySa cells treated with Compound 1 probed for various proteins of interest, including the SS18-SSX fusion protein, H2AK119ub, and Vinculin. FIG. 6B depicts a Western blot result example for YAMATO-SS cells treated with various doses of Compound 1 probed for various proteins of interest, including the SS18-SSX fusion protein, H2AK119ub, and Vinculin.

FIG. 7A depicts sequencing data derived from using CUT&RUN to detect localization of SS18-SSX fusion protein within gene bodies after treatment with DMSO (left) or Compound 1 (right). FIG. 7B depicts the localization of the SS18-SSX fusion protein, usually within 3 kb of the transcription start site (TSS) in SySa cells in direct target genes. Compound 1 treatment appears to attenuate this effect. FIG. 7C depicts sequencing data derived from using CUT&RUN to detect localization of H2AK119ub within gene bodies of SySa cells after treatment with DMSO (left) or Compound 1 (right). FIG. 7D depicts the localization of H2AK119ub, usually within 3 kb of the transcription start site (TSS) in SySa cells in direct target genes. Compound 1 treatment appears to attenuate this effect. FIG. 7E depicts the localization of SS18-SSX fusion protein, in SySa cells within SS18-SSX downstream genes like HOXA10 and SOX8. Compound 1 treatment appears to result in a decrease of SS18-SSX occupation of these genes. FIG. 7F depicts the localization of SS18-SSX fusion protein, in SySa cells within SS18-SSX repressed gene, GADD45. Compound 1 treatment appears to result in a decrease of SS18-SSX occupation of this gene and an increase in its expression.

FIG. 8A depicts a schematic showing an experiment timeline of the study's in vivo studies. The cartoon depicts time of Aska-SS or SYO1 cell injection, duration and frequency of Compound 1 treatment, and time of final tumor harvesting. FIG. 8B depicts the average tumor volumes of Aska-SS flank tumors over the duration of the experiment. FIG. 8C depicts the tumor weights for DMSO or Compound 1-treated Aska-SS flank tumors. FIG. 8D depicts the average tumor volumes of SYO1 flank tumors over the duration of the experiment. FIG. 8E depicts the tumor weights for DMSO or Compound 1-treated SYO1 flank tumors. FIG. 8F depicts the quantification of tumor cellularity in vehicle-treated and Compound 1-treated Aska-SS flank tumors. FIG. 8G depicts the quantification of tumor cellularity in vehicle-treated and Compound 1-treated SYO1 flank tumors. FIG. 8H depicts the quantification of Ki-67 staining in the vehicle and Compound 1-treated Aska-SS tumor sections from the periphery of the tumor. FIG. 8I depicts the quantification of Ki-67 staining in the vehicle and Compound 1-treated Aska-SS tumor sections from the center of the tumor. FIG. 8J depicts the quantification of Ki-67 staining in the vehicle and Compound 1-treated SYO1 tumor sections from the periphery of the tumor. FIG. 8K depicts the quantification of Ki-67 staining in the vehicle and Compound 1-treated SYO1 tumor sections from the center of the tumor.

### DETAILED DESCRIPTION

Disclosed herein are methods for treating a subject with cancer. The cancer may be a sarcoma. The sarcoma may be Synovial Sarcoma (SySa). The subject may be a patient. The method of treating may include using a small molecule SUMO E1 inhibitor. The small molecule SUMO E1 inhibitor may be Compound 1. The SUMO E1 inhibitor may have an effect on the fusion protein complex SS18-SSX. SSX may be SSX1, SSX2, SSX3 or SSX4. The effect on the SS18-SSX fusion complex may be inhibition of SUMOylation. The administration of the SUMO E1 inhibitor may inhibit proliferation or induce apoptosis of cancer cells, which may be SySa cells.

### Definitions

To facilitate an understanding of the present disclosure, a number of terms and phrases are defined below. Unless defined otherwise, all technical and scientific terms used herein have the same meaning as commonly understood by one of ordinary skill in the art to which this disclosure belongs.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “salt” refers to acid or base salts of a compound described herein having an acidic or basic moiety. Acid salts can be formed by combining a compound having a basic moiety with an acid. Base salts can be formed by combining a compound having an acidic moiety with a base. The salts may be prepared during the final isolation and purification of the compounds.

Pharmaceutically acceptable salts are disclosed in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+(C1-4alkyl)4 salts. This present disclosure also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The term “acid” refers to a compound that is an electron pair acceptor in an acid-base reaction. An acid can be an inorganic or organic acid.

The term “inorganic acid” refers to an acid that does not include a carbon bond. Inorganic acids can be a strong acid or a weak acid. Inorganic acids include, but are not limited to, hydrochloric acid, hydrobromic acid, sulfuric acid, phosphoric acid, nitric acid, sulfamic acid, perchloric acid, boric acid, fluorophosphoric acid, and metaphosphoric acid.

The term “organic acid” refers to an acid including at least one C—H bond, C—F bond, or C—C bond. Organic acids include, but are not limited to, acetic acid, trifluoroacetic acid, benzoic acid, citric acid, formic acid, fumaric acid, glycolic acid, isobutyric acid, lactic acid (DL), lactic acid (D), lactic acid (L), maleic acid, malonic acid, oxalic acid, propionic acid, salicylic acid, succinic acid, caprylic acid, L-(+)-tartaric acid, methanesulfonic acid, ethanesulfonic acid, benzenesulfonic acid, trifluoromethanesulfonic acid, p-toluenesulfonic acid, and camphor-10-sulfonic acid (+).

The term “base” refers to a compound that is an electron pair donor in an acid-base reaction. The base can be an inorganic base or an organic base.

The term “inorganic base” refers to a base that does not include at least one C—H bond and includes at least one alkali metal or alkaline earth metal. Examples of an inorganic base include, but are not limited to, sodium hydride, potassium hydride, lithium hydride, calcium hydride, barium carbonate, calcium carbonate, cesium carbonate, lithium carbonate, magnesium carbonate, potassium carbonate, sodium carbonate, cesium hydrogen carbonate, potassium hydrogen carbonate, sodium hydrogen carbonate, barium hydroxide, calcium hydroxide, cesium hydroxide, lithium hydroxide, magnesium hydroxide, potassium hydroxide, sodium hydroxide.

The term “organic base” refers to a base including at least one C—H bond (e.g., an amine base). In some embodiments, the amine base can be a primary, secondary, or tertiary amine. In some cases, compounds described herein coordinate with an amine base, such as, but not limited to, DIPEA, TEA, ethanolamine, diethanolamine, triethanolamine, tromethamine, meglumine, N-methylglucamine, dicyclohexylamine, and tris (hydroxymethyl) methylamine.

The term “isomers” refers to compounds with the same chemical formula, but which are structurally distinguishable. Certain compounds of the present disclosure possess asymmetric carbon atoms (optical centers) or double bonds; the racemates, diastereomers, geometric isomers and individual isomers are all intended to be encompassed within the scope of the present disclosure.

“Pharmaceutically acceptable” refers to a generally non-toxic, inert, and/or physiologically compatible composition or component of a composition. A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable, and/or can aid in the administration of an active agent to and absorption by a subject and can be included in the compositions of the present disclosure without causing a significant adverse toxicological effect on the patient.

As described above, the pharmaceutically acceptable compositions of the present disclosure additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and other techniques for the preparation thereof. Except insofar as any conventional carrier medium is incompatible with the compounds of the present disclosure, such as by producing any undesirable biological effect or otherwise interacting in a deleterious manner with any other component(s) of the pharmaceutically acceptable composition, its use is contemplated to be within the scope of the present disclosure. Some examples of materials which can serve as pharmaceutically acceptable carriers include, but are not limited to, ion exchangers; alumina; aluminum stearate; lecithin; serum proteins, such as human serum albumin; buffer substances such as phosphates (including but not limited to phosphate buffer solutions), glycine, sorbic acid, or potassium sorbate; partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica; magnesium trisilicate; polyvinyl pyrrolidone; polyacrylates; waxes; polyethylene-polyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “population of cells” (including TILs) can mean a number of cells that share common traits. In general, populations generally range from 1×10⁶ to 1×10¹⁰ in number.

As used herein, the term “cancer” refers to a cellular disorder characterized by uncontrolled or dysregulated cell proliferation, decreased cellular differentiation, inappropriate ability to invade surrounding tissue, and/or ability to establish new growth at ectopic sites. The term “cancer” includes, but is not limited to, solid tumors and bloodborne tumors (hematologic malignancies). The term “cancer” encompasses diseases of skin, tissues, organs, bone, cartilage, blood, and vessels. The term “cancer” further encompasses primary and metastatic cancers.

The term “treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating, improving a patient's physical or mental well-being. The treatment or amelioration of symptoms can be based on objective or subjective parameters.

The term “treatment” and “treating” and the like can also be used to mean obtaining a desired pharmacological and physiological effect. The effect may be prophylactic in terms of preventing or partially preventing a disease, symptom, or condition thereof and/or may be therapeutic in terms of a partial or complete cure of a disease, condition, symptom, or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it such as a preventive early asymptomatic intervention; (b) inhibiting the disease, i.e., arresting its development; or relieving the disease, i.e., causing regression of the disease and/or its symptoms or conditions such as improvement or remediation of damage.

Effective treatment can be indicated by a decrease in number or percentage of cancer cells, inhibiting proliferation of cancer cells, shrinking or arresting of tumor size, causing apoptosis of cancer cells, causing necrosis of cancer cells, prevention of cancer metastasis, cancer remission, or other metric indicating a reduction in cancer cells.

The term “patient” or “subject” in need thereof refers to a living organism suffering from or prone to a condition that can be treated by administration of a pharmaceutical composition as provided herein. The term “subject” as used herein refers to mammals. For examples, mammals contemplated by the present disclosure include human, primates, domesticated animals such as cattle, sheep, pigs, horses, laboratory rodents, other pets and the like.

The expression “dosage” as used herein refers to a physically discrete unit of agent appropriate for the patient to be treated. It will be understood, however, that the total per treatment usage of the compounds and compositions of the present disclosure will be decided by the attending physician within the scope of sound medical judgment. The specific effective dose level for any particular patient or organism will depend upon a variety of factors including the disease being treated and the severity of the disease; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors disclosed in the medical arts.

The term “adult” refers to a human patient or subject at or above about 22 years of age.

The term “pediatric” refers to a human patient or subject below about 22 years of age and includes: (1) neonates, between birth and the first 28 days of life; (2) infants, from 29 days to less than 2 years of age; (3) children, from 2 to less than 12 years of age; and (4) adolescence, from 12 through 21 years of age (up to but not including the 22nd birthday).

The term “administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

The terms “effective amount” or “therapeutically effective amount” or “therapeutic effect” refer to an amount of a therapeutic effective to “treat” a disease or disorder in a subject or mammal. The therapeutically effective amount of a drug has a therapeutic effect and as such can prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The singular form “a,” “an,” and “the” include plural references unless the context clearly dictates otherwise. For example, the term “a cell” includes one or more cells, including mixtures thereof. “Item 1 and/or Item 2” is used herein to include all of the following alternatives: “Item 1”, “Item 2”, “Item 1 or Item 2”, and “Item 1 and Item 2.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

### Pharmaceutical Compositions

As described above, the pharmaceutically acceptable compositions of the present disclosure additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and other techniques for the preparation thereof. Except insofar as any conventional carrier medium is incompatible with the compounds of the present disclosure, such as by producing any undesirable biological effect or otherwise interacting in a deleterious manner with any other component(s) of the pharmaceutically acceptable composition, its use is contemplated to be within the scope of the present disclosure. Some examples of materials which can serve as pharmaceutically acceptable carriers include, but are not limited to, ion exchangers; alumina; aluminum stearate; lecithin; serum proteins, such as human serum albumin; buffer substances such as phosphates (including but not limited to phosphate buffer solutions), glycine, sorbic acid, or potassium sorbate; partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica; magnesium trisilicate; polyvinyl pyrrolidone; polyacrylates; waxes; polyethylene-polyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water, isotonic saline; Ringer's solution; ethyl alcohol, and non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds and compositions, according to the method of the present disclosure, may be administered using any amount and any route of administration effective for treating the disease. The exact amount required may vary from subject to subject, depending on the species, age, and general condition of the subject, the severity of the infection, the particular agent, its mode of administration, and the like. The compounds of the present disclosure are frequently formulated in dosage unit form for ease of administration and uniformity of dosage.

The pharmaceutically acceptable compositions of the present disclosure can be administered to humans and other animals orally, rectally, parenterally, intracisternally, intravaginally, intraperitoneally, topically (as by powders, ointments, lotions, or drops), buccally, as an oral or nasal spray, or the like, depending on the severity of the infection being treated. In some embodiments, the compound or pharmaceutical composition described herein is administered intravenous. In certain embodiments, the compounds of the present disclosure may be administered orally, intravenously, or parenterally at dosage levels of about 0.01 mg to about 150 mg, for instance from about 0.5 mg to about 75 mg, per subject per day, one or more times a day, to obtain the desired therapeutic effect.

Liquid dosage forms for oral administration include, but are not limited to, pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (for instance, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, and tablets, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, and granules can be prepared with coatings and shells such as enteric coatings and other coatings.

The active compounds can also be in micro-encapsulated form with one or more excipients. The solid dosage forms of tablets, dragees, capsules, and granules can be prepared with coatings and shells such as enteric coatings, release controlling coatings and other coatings in the pharmaceutical formulating art. In such solid dosage forms the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and the dosage forms may also comprise buffering agents. They may optionally contain opacifying agents and can also be of a composition that they release the active ingredient(s) only, or for instance in a certain part of the intestinal tract, optionally, in a delayed manner. Examples of embedding compositions that can be used include polymeric substances and waxes.

In some embodiments, the compounds of the present disclosure or a pharmaceutical composition thereof is administered in conjunction with an anticancer agent. As used herein, the term “anticancer agent” refers to any agent that is administered to a subject with cancer for purposes of treating the cancer. Combination therapy includes administration of the therapeutic agents concurrently or sequentially. Alternatively, the therapeutic agents can be combined into one composition which is administered to the patient.

### Therapeutic Combination Administration

Readouts of in vitro and in vivo pooled CRISPR/Cas9 screens to identify common genes essential to SySa proliferation can include, but are not limited to, SUMO2, PIAS1, MDM4, RPL13, BICDL1, USP7, WDR5, LGALS7B, CSH2, FRG2, SLC16A13, RASA4B, SRSF2, KAT2A, TNS2, TPS3I13, SUMO1P3, KAT2A and HIST3H2BB.These genes are involved in various biochemical pathways, including: chromosome organization, WNT signaling, BAF complex, and PRCI activity which are known dependencies in synovial sarcoma. Novel biological pathways and protein complexes identified included the SUMO2-UBE2I complex, the SAGA, and the synaptojanin complex

In some embodiments, the compounds of the present disclosure are used in combination with other therapeutic agents. In some embodiments, a compound of the present disclosure is administered in conjunction with another therapeutic agent wherein the therapeutic agent is chemotherapeutic agents, radiotherapy, or immunotherapy.

Non-limiting examples of chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea). In some embodiments, the chemotherapeutic agent is ifosfamide or doxorubicin. Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., pazopanib, imatinib mesylate and gefitinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, and bevacizumab); and other inhibitors of proteins or enzymes that are upregulated, over-expressed or activated in cancers, the inhibition of which downregulates cell replication. In certain embodiments, a compound of the present disclosure is administered in conjunction with a proteasome inhibitor. In certain embodiments, a compound of the present disclosure is administered in conjunction with a receptor tyrosine kinase inhibitor. In certain embodiments, a compound of the present disclosure is administered in conjunction with pazopanib.

Another aspect of the present disclosure relates to inhibiting SUMO activating enzyme (SAE) activity in a biological sample or a patient, which method comprises administering to the patient, or contacting said biological sample with a compound described herein, or a composition comprising said compound. The term “biological sample,” as used herein, generally includes in vivo, in vitro, and ex vivo materials, and also includes, without limitation, cell cultures or extracts thereof, biopsied material obtained from a mammal or extracts thereof; and blood, saliva, urine, feces, semen, tears, or other body fluids or extracts thereof.

### Methods of Treatment

In one embodiment, provided herein is a method of reducing proliferation in a population of cancer cells. In some aspects, the cancer cells can be soft tissue sarcoma cells. In some cases, the soft tissue sarcoma cells can be liposarcoma cells. In some cases, the soft tissue sarcoma cells can be angiosarcoma cells. In some cases, the soft tissue sarcoma cells can be epithelioid sarcoma cells. In some cases, the soft tissue sarcoma cells can be leiomyosarcoma cells. In some cases, the soft tissue sarcoma cells can be synovial sarcoma cells. In some cases, the soft tissue sarcoma cells can be gastrointestinal stromal cancer cells. In some cases, the soft tissue sarcoma cells can be fibrosarcoma cells. In some cases, the soft tissue sarcoma cells can be clear cell sarcoma cells. In some cases, the soft tissue sarcoma cells can be rhabdomyosarcoma cells. In some cases, the soft tissue sarcoma cells can be dermatofibrosarcoma cells. In some cases, the soft tissue sarcoma cells can be undifferentiated pleomorphic sarcoma cells. In some cases, the soft tissue sarcoma cells can be fibrous histiocytoma cells. In some cases, the soft tissue sarcoma cells can be Ewing sarcoma cells. In some cases, the soft tissue sarcoma cells can be fibromatosis cells. In some cases, the soft tissue sarcoma cells can be ganglioneuroblastoma cells. In some cases, the soft tissue sarcoma cells can be chondrosarcoma cells. In some cases, the soft tissue sarcoma cells can be myxoid chondrosarcoma cells. In some cases, the soft tissue sarcoma cells can be hermangioendothelioma cells. In some cases, the soft tissue sarcoma cells can be hemangioma cells. In some cases, the soft tissue sarcoma cells can be PEComa cells. In some cases, the soft tissue sarcoma cells can be spindle cell sarcoma cells.

In some aspects, the method can reduce proliferation in a population of synovial sarcoma cell lines. In some cases, the method described herein can reduce proliferation in a population of HS-SY-II cells. In some cases, the method described herein can reduce proliferation in a population of SYO1 cells. In some cases, the method described herein can reduce proliferation in a population of FUJI cells. In some cases, the method described herein can reduce proliferation in a population of CME1 cells. In some cases, the method described herein can reduce proliferation in a population of Aska-SS cells. In some cases, the method described herein can reduce proliferation in a population of Yamato-SS cells. In some cases, the method described herein can reduce proliferation in a population of SW982 cells. In some cases, the method described herein can reduce proliferation in a population of SS1A cells. In some cases, the method described herein can reduce proliferation in a population of 1273/99 cells. In some cases, the method described herein can reduce proliferation in a population of 716 SS MNV cells. In some cases, the method described herein can reduce proliferation in a population of A-1095 cells. In some cases, the method described herein can reduce proliferation in a population of A2243 cells. In some cases, the method described herein can reduce proliferation in a population of FU-SY-1 cells. In some cases, the method described herein can reduce proliferation in a population of CNIO BL cells. In some cases, the method described herein can reduce proliferation in a population of GM07166VA7 cells. In some cases, the method described herein can reduce proliferation in a population of GM07166VA7-NBS1 cells. In some cases, the method described herein can reduce proliferation in a population of GUSS-1 cells. In some cases, the method described herein can reduce proliferation in a population of GUSS-2 cells. In some cases, the method described herein can reduce proliferation in a population of GUSS-3 cells. In some cases, the method described herein can reduce proliferation in a population of GUSS-3b cells. In some cases, the method described herein can reduce proliferation in a population of Hs 192.T cells. In some cases, the method described herein can reduce proliferation in a population of Hs 197.T cells. In some cases, the method described herein can reduce proliferation in a population of HS 431.T cells. In some cases, the method described herein can reduce proliferation in a population of Hs 701. T cells. In some cases, the method described herein can reduce proliferation in a population of HS-SY-II cells. In some cases, the method described herein can reduce proliferation in a population of hSS-005R cells. In some cases, the method described herein can reduce proliferation in a population of HSS-84 cells. In some cases, the method described herein can reduce proliferation in a population of ICR-SS-1 cells. In some cases, the method described herein can reduce proliferation in a population of KU-SS-1 cells. In some cases, the method described herein can reduce proliferation in a population of NCC-SSI-C1 cells. In some cases, the method described herein can reduce proliferation in a population of NCC-SS2-C1 cells. In some cases, the method described herein can reduce proliferation in a population of NCC-SS3-C1 cells. In some cases, the method described herein can reduce proliferation in a population of NCC-SS4-C1 cells. In some cases, the method described herein can reduce proliferation in a population of NCC-SSS-C1 cells. In some cases, the method described herein can reduce proliferation in a population of PDSS-26 cells. In some cases, the method described herein can reduce proliferation in a population of RIT-3 cells. In some cases, the method described herein can reduce proliferation in a population of SCS214 cells. In some cases, the method described herein can reduce proliferation in a population of SN-SY-1 cells. In some cases, the method described herein can reduce proliferation in a population of STS255 cells. In some cases, the method described herein can reduce proliferation in a population of STSAR-198 cells. In some cases, the method described herein can reduce proliferation in a population of STSAR-84 cells. In some cases, the method described herein can reduce proliferation in a population of SW1045 cells. In some cases, the method described herein can reduce proliferation in a population of SYN-1 cells. In some cases, the method described herein can reduce proliferation in a population of SYNb-1 cells. In some cases, the method described herein can reduce proliferation in a population of SYNb-2 cells. In some cases, the method described herein can reduce proliferation in a population of YaFUSS cells. In some cases, the method described herein can reduce proliferation in one or more populations of HS-SY-II cells, SYO1 cells, FUJI cells, CME1 cells, Aska-SS cells, Yamato-SS cells, SW982 cells, SS1A cells, or any combination thereof. In some cases, the method described herein can reduce proliferation in one or more populations of HS-SY-II cells, SYO1 cells, FUJI cells, CME1 cells, Aska-SS cells, Yamato-SS cells, SW982 cells, SS1A cells, 1273/99 cells, 716 SS MNV cells, A-1095 cells, A2243 cells, FU-SY-1 cells, CNIO BL cells, GM07166VA7 cells, GM07166VA7-NBS1 cells, GUSS-1 cells, GUSS-2 cells, GUSS-3 cells, GUSS-3b cells, Hs 192. T cells, Hs 197. T cells, HS 431.T cells, Hs 701.T cells, HS-SY-III cells, hSS-005R cells, HSS-84 cells, ICR-SS-1 cells, KU-SS-1 cells, NCC-SSI-C1 cells, NCC-SS2-C1 cells, NCC-SS3-C1 cells, NCC-SS4-C1 cells, NCC-SS5-C1 cells, OST 10 PT cells, OST 7 PT cells, OST 8 PT cells, PDSS-26 cells, RIT-3 cells, SCS214 cells, SN-SY-1 cells, STS255 cells, STSAR-198 cells, STSAR-84 cells, SW1045 cells, SYN-1 cells, SYNb-1 cells, SYNb-2 cells, YaFUSS cells, or any combination thereof.

In some aspects, the method can reduce proliferation of cancer cells. In some aspects, the method can reduce proliferation of sarcoma cells. In some aspects, the method can reduce proliferation of synovial sarcoma cells.

In some aspects, the method can induce apoptosis of cancer cells. In some aspects, the method can induce apoptosis of sarcoma cells. In some aspects, the method can induce apoptosis of synovial sarcoma cells.

In some aspects, the method can induce apoptosis in a population of synovial sarcoma cell lines. In some cases, the method described herein can induce apoptosis in a population of HS-SY-II cells. In some cases, the method described herein can induce apoptosis in a population of SYO1 cells. In some cases, the method described herein can induce apoptosis in a population of FUJI cells. In some cases, the method described herein can induce apoptosis in a population of CME1 cells. In some cases, the method described herein can induce apoptosis in a population of Aska-SS cells. In some cases, the method described herein can induce apoptosis in a population of Yamato-SS cells. In some cases, the method described herein can induce apoptosis in a population of SW982 cells. In some cases, the method described herein can induce apoptosis in a population of SS1A cells. In some cases, the method described herein can induce apoptosis in a population of 1273/99 cells. In some cases, the method described herein can induce apoptosis in a population of 716 SS MNV cells. In some cases, the method described herein can induce apoptosis in a population of A-1095 cells. In some cases, the method described herein can induce apoptosis in a population of A2243 cells. In some cases, the method described herein can induce apoptosis in a population of FU-SY-1 cells. In some cases, the method described herein can induce apoptosis in a population of CNIO BL cells. In some cases, the method described herein can induce apoptosis in a population of GM07166VA7 cells. In some cases, the method described herein can induce apoptosis in a population of GM07166VA7-NBS1 cells. In some cases, the method described herein can induce apoptosis in a population of GUSS-1 cells. In some cases, the method described herein can induce apoptosis in a population of GUSS-2 cells. In some cases, the method described herein can induce apoptosis in a population of GUSS-3 cells. In some cases, the method described herein can induce apoptosis in a population of GUSS-3b cells. In some cases, the method described herein can induce apoptosis in a population of Hs 192.T cells. In some cases, the method described herein can induce apoptosis in a population of Hs 197.T cells. In some cases, the method described herein can induce apoptosis in a population of HS 431.T cells. In some cases, the method described herein can induce apoptosis in a population of Hs 701. T cells. In some cases, the method described herein can induce apoptosis in a population of HS-SY-III cells. In some cases, the method described herein can induce apoptosis in a population of hSS-00SR cells. In some cases, the method described herein can induce apoptosis in a population of HSS-84 cells. In some cases, the method described herein can induce apoptosis in a population of ICR-SS-1 cells. In some cases, the method described herein can induce apoptosis in a population of KU-SS-1 cells. In some cases, the method described herein can induce apoptosis in a population of NCC-SSI-C1 cells. In some cases, the method described herein can induce apoptosis in a population of NCC-SS2-C1 cells. In some cases, the method described herein can induce apoptosis in a population of NCC-SS3-C1 cells. In some cases, the method described herein can induce apoptosis in a population of NCC-SS4-C1 cells. In some cases, the method described herein can induce apoptosis in a population of NCC-SSS-C1 cells. In some cases, the method described herein can induce apoptosis in a population of OST 10 PT cells. In some cases, the method described herein can induce apoptosis in a population of OST 7 PT cells. In some cases, the method described herein can induce apoptosis in a population of OST 8 PT cells. In some cases, the method described herein can induce apoptosis in a population of PDSS-26 cells. In some cases, the method described herein can induce apoptosis in a population of RIT-3 cells. In some cases, the method described herein can induce apoptosis in a population of SCS214 cells. In some cases, the method described herein can induce apoptosis in a population of SN-SY-1 cells. In some cases, the method described herein can induce apoptosis in a population of STS255 cells. In some cases, the method described herein can induce apoptosis in a population of STSAR-198 cells. In some cases, the method described herein can induce apoptosis in a population of STSAR-84 cells. In some cases, the method described herein can induce apoptosis in a population of SW1045 cells. In some cases, the method described herein can induce apoptosis in a population of SYN-1 cells. In some cases, the method described herein can induce apoptosis in a population of SYNb-1 cells. In some cases, the method described herein can induce apoptosis in a population of SYNb-2 cells. In some cases, the method described herein can induce apoptosis in a population of YaFUSS cells. In some cases, the method described herein can induce apoptosis in one or more populations of HS-SY-II cells, SYO1 cells, FUJI cells, CME1 cells, Aska-SS cells, Yamato-SS cells, SW982 cells, SS1A cells, or any combination thereof. In some cases, the method described herein can induce apoptosis in one or more populations of HS-SY-II cells, SYO1 cells, FUJI cells, CME1 cells, Aska-SS cells, Yamato-SS cells, SW982 cells, SS1A cells, 1273/99 cells, 716 SS MNV cells, A-1095 cells, A2243 cells, FU-SY-1 cells, CNIO BL cells, GM07166VA7 cells, GM07166VA7-NBS1 cells, GUSS-1 cells, GUSS-2 cells, GUSS-3 cells, GUSS-3b cells, Hs 192. T cells, Hs 197.T cells, HS 431.T cells, Hs 701. T cells, HS-SY-III cells, hSS-005R cells, HSS-84 cells, ICR-SS-1 cells, KU-SS-1 cells, NCC-SSI-C1 cells, NCC-SS2-C1 cells, NCC-SS3-C1 cells, NCC-SS4-C1 cells, NCC-SS5-C1 cells, OST 10 PT cells, OST 7 PT cells, OST 8 PT cells, PDSS-26 cells, RIT-3 cells, SCS214 cells, SN-SY-1 cells, STS255 cells, STSAR-198 cells, STSAR-84 cells, SW1045 cells, SYN-1 cells, SYNb-1 cells, SYNb-2 cells, YaFUSS cells, or any combination thereof.

In some aspects, the method can induce necrosis of cancer cells. In some aspects, the method can induce necrosis of sarcoma cells. In some aspects, the method can induce necrosis of synovial sarcoma cells.

In some aspects, the method can induce necrosis in a population of synovial sarcoma cell lines. In some cases, the method described herein can induce necrosis in a population of HS-SY-II cells. In some cases, the method described herein can induce necrosis in a population of SYO1 cells. In some cases, the method described herein can induce necrosis in a population of FUJI cells. In some cases, the method described herein can induce necrosis in a population of CMEI cells. In some cases, the method described herein can induce necrosis in a population of Aska-SS cells. In some cases, the method described herein can induce necrosis in a population of Yamato-SS cells. In some cases, the method described herein can induce necrosis in a population of SW982 cells. In some cases, the method described herein can induce necrosis in a population of SS1A cells. In some cases, the method described herein can induce necrosis in a population of 1273/99 cells. In some cases, the method described herein can induce necrosis in a population of 716 SS MNV cells. In some cases, the method described herein can induce necrosis in a population of A-1095 cells. In some cases, the method described herein can induce necrosis in a population of A2243 cells. In some cases, the method described herein can induce necrosis in a population of FU-SY-1 cells. In some cases, the method described herein can induce necrosis in a population of CNIO BL cells. In some cases, the method described herein can induce necrosis in a population of GM07166VA7 cells. In some cases, the method described herein can induce necrosis in a population of GM07166VA7-NBS1 cells. In some cases, the method described herein can induce necrosis in a population of GUSS-1 cells. In some cases, the method described herein can induce necrosis in a population of GUSS-2 cells. In some cases, the method described herein can induce necrosis in a population of GUSS-3 cells. In some cases, the method described herein can induce necrosis in a population of GUSS-3b cells. In some cases, the method described herein can induce necrosis in a population of Hs 192.T cells. In some cases, the method described herein can induce necrosis in a population of Hs 197.T cells. In some cases, the method described herein can induce necrosis in a population of HS 431. T cells. In some cases, the method described herein can induce necrosis in a population of Hs 701.T cells. In some cases, the method described herein can induce necrosis in a population of HS-SY-III cells. In some cases, the method described herein can induce necrosis in a population of hSS-005R cells. In some cases, the method described herein can induce necrosis in a population of HSS-84 cells. In some cases, the method described herein can induce necrosis in a population of ICR-SS-1 cells. In some cases, the method described herein can induce necrosis in a population of KU-SS-1 cells. In some cases, the method described herein can induce necrosis in a population of NCC-SS1-C1 cells. In some cases, the method described herein can induce necrosis in a population of NCC-SS2-C1 cells. In some cases, the method described herein can induce necrosis in a population of NCC-SS3-C1 cells. In some cases, the method described herein can induce necrosis in a population of NCC-SS4-C1 cells. In some cases, the method described herein can induce necrosis in a population of NCC-SS5-C1 cells. In some cases, the method described herein can induce necrosis in a population of OST 10 PT cells. In some cases, the method described herein can induce necrosis in a population of OST 7 PT cells. In some cases, the method described herein can induce necrosis in a population of OST 8 PT cells. In some cases, the method described herein can induce necrosis in a population of PDSS-26 cells. In some cases, the method described herein can induce necrosis in a population of RIT-3 cells. In some cases, the method described herein can induce necrosis in a population of SCS214 cells. In some cases, the method described herein can induce necrosis in a population of SN-SY-1 cells. In some cases, the method described herein can induce necrosis in a population of STS255 cells. In some cases, the method described herein can induce necrosis in a population of STSAR-198 cells. In some cases, the method described herein can induce necrosis in a population of STSAR-84 cells. In some cases, the method described herein can induce necrosis in a population of SW 1045 cells. In some cases, the method described herein can induce necrosis in a population of SYN-1 cells. In some cases, the method described herein can induce necrosis in a population of SYNb-1 cells. In some cases, the method described herein can induce necrosis in a population of SYNb-2 cells. In some cases, the method described herein can induce necrosis in a population of YaFUSS cells. In some cases, the method described herein can induce necrosis in one or more populations of HS-SY-II cells, SYO1 cells, FUJI cells, CME1 cells, Aska-SS cells, Yamato-SS cells, SW982 cells, SS1A cells, or any combination thereof. In some cases, the method described herein can induce necrosis in one or more populations of HS-SY-II cells, SYO1 cells, FUJI cells, CMEI cells, Aska-SS cells, Yamato-SS cells, SW982 cells, SS1A cells, 1273/99 cells, 716 SS MNV cells, A-1095 cells, A2243 cells, FU-SY-1 cells, CNIO BL cells, GM07166VA7 cells, GM07166VA7-NBS1 cells, GUSS-1 cells, GUSS-2 cells, GUSS-3 cells, GUSS-3b cells, Hs 192. T cells, Hs 197.T cells, HS 431.T cells, Hs 701.T cells, HS-SY-III cells, hSS-005R cells, HSS-84 cells, ICR-SS-1 cells, KU-SS-1 cells, NCC-SS1-C1 cells, NCC-SS2-C1 cells, NCC-SS3-C1 cells, NCC-SS4-C1 cells, NCC-SSS-C1 cells, OST 10 PT cells, OST 7 PT cells, OST 8 PT cells, PDSS-26 cells, RIT-3 cells, SCS214 cells, SN-SY-1 cells, STS255 cells, STSAR-198 cells, STSAR-84 cells, SW1045 cells, SYN-1 cells, SYNb-1 cells, SYNb-2 cells, YaFUSS cells, or any combination thereof.

In some aspects, the method can reduce tumor (e.g., xenograft tumors) volumes. In some aspects, the method can reduce volumes of tumors comprising sarcoma cells. In some aspects, the method can reduce volumes of tumors comprising synovial sarcoma cells. In some cases, the method described herein can reduce volumes of tumors comprising HIS-SY-II cells. In some cases, the method described herein can reduce volumes of tumors comprising SYO1 cells. In some cases, the method described herein can reduce volumes of tumors comprising FUJI cells. In some cases, the method described herein can reduce volumes of tumors comprising CME1 cells. In some cases, the method described herein can reduce volumes of tumors comprising Aska-SS cells. In some cases, the method described herein can reduce volumes of tumors comprising Yamato-SS cells. In some cases, the method described herein can reduce volumes of tumors comprising SW982 cells. In some cases, the method described herein can reduce volumes of tumors comprising SS1A cells. In some cases, the method described herein can reduce volumes of tumors comprising 1273/99 cells. In some cases, the method described herein can reduce volumes of tumors comprising 716 SS MNV cells. In some cases, the method described herein can reduce volumes of tumors comprising A-1095 cells. In some cases, the method described herein can reduce volumes of tumors comprising A2243 cells. In some cases, the method described herein can reduce volumes of tumors comprising FU-SY-1 cells. In some cases, the method described herein can reduce volumes of tumors comprising CNIO BL cells. In some cases, the method described herein can reduce volumes of tumors comprising cells. In some cases, the method described herein can reduce volumes of tumors comprising GM07166VA7-NBS1 cells. In some cases, the method described herein can reduce volumes of tumors comprising GUSS-1 cells. In some cases, the method described herein can reduce volumes of tumors comprising GUSS-2 cells. In some cases, the method described herein can reduce volumes of tumors comprising GUSS-3 cells. In some cases, the method described herein can reduce volumes of tumors comprising GUSS-3b cells. In some cases, the method described herein can reduce volumes of tumors comprising Hs 192.T cells. In some cases, the method described herein can reduce volumes of tumors comprising HS 197.T cells. In some cases, the method described herein can reduce volumes of tumors comprising HS 431.T cells. In some cases, the method described herein can reduce volumes of tumors comprising HS 701. T cells. In some cases, the method described herein can reduce volumes of tumors comprising HS-SY-II cells. In some cases, the method described herein can reduce volumes of tumors comprising hSS-005R cells. In some cases, the method described herein can reduce volumes of tumors comprising HSS-84 cells. In some cases, the method described herein can reduce volumes of tumors comprising ICR-SS-1 cells. In some cases, the method described herein can reduce volumes of tumors comprising KU-SS-1 cells. In some cases, the method described herein can reduce volumes of tumors comprising NCC-SS1-C1 cells. In some cases, the method described herein can reduce volumes of tumors comprising NCC-SS2-C1 cells. In some cases, the method described herein can reduce volumes of tumors comprising NCC-SS3-C1 cells. In some cases, the method described herein can reduce volumes of tumors comprising NCC-SS4-C1 cells. In some cases, the method described herein can reduce volumes of tumors comprising NCC-SS5-C1 cells. In some cases, the method described herein can reduce volumes of tumors comprising OST 10 PT cells. In some cases, the method described herein can reduce volumes of tumors comprising OST 7 PT cells. In some cases, the method described herein can reduce volumes of tumors comprising OST 8 PT cells. In some cases, the method described herein can reduce volumes of tumors comprising PDSS-26 cells. In some cases, the method described herein can reduce volumes of tumors comprising RIT-3 cells. In some cases, the method described herein can reduce volumes of tumors comprising SCS214 cells. In some cases, the method described herein can reduce volumes of tumors comprising SN-SY-1 cells. In some cases, the method described herein can reduce volumes of tumors comprising STS255 cells. In some cases, the method described herein can reduce volumes of tumors comprising STSAR-198 cells. In some cases, the method described herein can reduce volumes of tumors comprising STSAR-84 cells. In some cases, the method described herein can reduce volumes of tumors comprising SW1045 cells. In some cases, the method described herein can reduce volumes of tumors comprising SYN-1 cells. In some cases, the method described herein can reduce volumes of tumors comprising SYNb-1 cells. In some cases, the method described herein can reduce volumes of tumors comprising SYNb-2 cells. In some cases, the method described herein can reduce volumes of tumors comprising YaFUSS cells. In some cases, the method described herein can reduce volumes of tumors comprising HS-SY-II cells, SYO1 cells, FUJI cells, CME1 cells, Aska-SS cells, Yamato-SS cells, SW982 cells, SS1A cells, or any combination thereof. In some cases, the method described herein can reduce volumes of tumors comprising HS-SY-II cells, SYO1 cells, FUJI cells, CMEI cells, Aska-SS cells, Yamato-SS cells, SW982 cells, SS1A cells, 1273/99 cells, 716 SS MNV cells, A-1095 cells, A2243 cells, FU-SY-1 cells, CNIO BL cells, GM07166VA7 cells, GM07166VA7-NBS1 cells, GUSS-1 cells, GUSS-2 cells, GUSS-3 cells, GUSS-3b cells, Hs 192.T cells, Hs 197.T cells, HS 431. T cells, Hs 701.T cells, HS-SY-III cells, hSS-005R cells, HSS-84 cells, ICR-SS-1 cells, KU-SS-1 cells, NCC-SS1-C1 cells, NCC-SS2-C1 cells, NCC-SS3-C1 cells, NCC-SS4-C1 cells, NCC-SSS-C1 cells, OST 10 PT cells, OST 7 PT cells, OST 8 PT cells, PDSS-26 cells, RIT-3 cells, SCS214 cells, SN-SY-1 cells, STS255 cells, STSAR-198 cells, STSAR-84 cells, SW1045 cells, SYN-1 cells, SYNb-1 cells, SYNb-2 cells, YaFUSS cells, or any combination thereof.

In some aspects, the method can reduce tumor (e.g., xenograft tumors) weights. In some aspects, the method can reduce weights of tumors comprising sarcoma cells. In some aspects, the method can reduce weights of tumors comprising synovial sarcoma cells. In some cases, the method described herein can reduce weights of tumors comprising HS-SY-II cells. In some cases, the method described herein can reduce weights of tumors comprising SYO1 cells. In some cases, the method described herein can reduce weights of tumors comprising FUJI cells. In some cases, the method described herein can reduce weights of tumors comprising CME1 cells. In some cases, the method described herein can reduce weights of tumors comprising Aska-SS cells. In some cases, the method described herein can reduce weights of tumors comprising Yamato-SS cells. In some cases, the method described herein can reduce weights of tumors comprising SW982 cells. In some cases, the method described herein can reduce weights of tumors comprising SS1A cells. In some cases, the method described herein can reduce weights of tumors comprising 1273/99 cells. In some cases, the method described herein can reduce weights of tumors comprising 716 SS MNV cells. In some cases, the method described herein can reduce weights of tumors comprising A-1095 cells. In some cases, the method described herein can reduce weights of tumors comprising A2243 cells. In some cases, the method described herein can reduce weights of tumors comprising FU-SY-1 cells. In some cases, the method described herein can reduce weights of tumors comprising CNIO BL cells. In some cases, the method described herein can reduce weights of tumors comprising cells. In some cases, the method described herein can reduce weights of tumors comprising GM07166VA7-NBS1 cells. In some cases, the method described herein can reduce weights of tumors comprising GUSS-1 cells. In some cases, the method described herein can reduce weights of tumors comprising GUSS-2 cells. In some cases, the method described herein can reduce weights of tumors comprising GUSS-3 cells. In some cases, the method described herein can reduce weights of tumors comprising GUSS-3b cells. In some cases, the method described herein can reduce weights of tumors comprising Hs 192.T cells. In some cases, the method described herein can reduce weights of tumors comprising HIS 197.T cells. In some cases, the method described herein can reduce weights of tumors comprising HS 431.T cells. In some cases, the method described herein can reduce weights of tumors comprising HS 701. T cells. In some cases, the method described herein can reduce weights of tumors comprising HS-SY-II cells. In some cases, the method described herein can reduce weights of tumors comprising hSS-005R cells. In some cases, the method described herein can reduce weights of tumors comprising HSS-84 cells. In some cases, the method described herein can reduce weights of tumors comprising ICR-SS-1 cells. In some cases, the method described herein can reduce weights of tumors comprising KU-SS-1 cells. In some cases, the method described herein can reduce weights of tumors comprising NCC-SS1-C1 cells. In some cases, the method described herein can reduce weights of tumors comprising NCC-SS2-C1 cells. In some cases, the method described herein can reduce weights of tumors comprising NCC-SS3-C1 cells. In some cases, the method described herein can reduce weights of tumors comprising NCC-SS4-C1 cells. In some cases, the method described herein can reduce weights of tumors comprising NCC-SSS-CI cells. In some cases, the method described herein can reduce weights of tumors comprising OST 10 PT cells. In some cases, the method described herein can reduce weights of tumors comprising OST 7 PT cells. In some cases, the method described herein can reduce weights of tumors comprising OST 8 PT cells. In some cases, the method described herein can reduce weights of tumors comprising PDSS-26 cells. In some cases, the method described herein can reduce weights of tumors comprising RIT-3 cells. In some cases, the method described herein can reduce weights of tumors comprising SCS214 cells. In some cases, the method described herein can reduce weights of tumors comprising SN-SY-1 cells. In some cases, the method described herein can reduce weights of tumors comprising STS255 cells. In some cases, the method described herein can reduce weights of tumors comprising STSAR-198 cells. In some cases, the method described herein can reduce weights of tumors comprising STSAR-84 cells. In some cases, the method described herein can reduce weights of tumors comprising SW1045 cells. In some cases, the method described herein can reduce weights of tumors comprising SYN-1 cells. In some cases, the method described herein can reduce weights of tumors comprising SYNb-1 cells. In some cases, the method described herein can reduce weights of tumors comprising SYNb-2 cells. In some cases, the method described herein can reduce weights of tumors comprising YaFUSS cells. In some cases, the method described herein can reduce weights of tumors comprising HS-SY-II cells, SYO1 cells, FUJI cells, CME1 cells, Aska-SS cells, Yamato-SS cells, SW982 cells, SS1A cells, or any combination thereof. In some cases, the method described herein can reduce weights of tumors comprising HS-SY-II cells, SYO1 cells, FUJI cells, CME1 cells, Aska-SS cells, Yamato-SS cells, SW982 cells, SS1A cells, 1273/99 cells, 716 SS MNV cells, A-1095 cells, A2243 cells, FU-SY-1 cells, CNIO BL cells, GM07166VA7 cells, GM07166VA7-NBS1 cells, GUSS-1 cells, GUSS-2 cells, GUSS-3 cells, GUSS-3b cells, Hs 192.T cells, Hs 197.T cells, HS 431.T cells, Hs 701. T cells, HS-SY-III cells, hSS-005R cells, HSS-84 cells, ICR-SS-1 cells, KU-SS-1 cells, NCC-SSI-C1 cells, NCC-SS2-C1 cells, NCC-SS3-C1 cells, NCC-SS4-C1 cells, NCC-SS5-C1 cells, OST 10 PT cells, OST 7 PT cells, OST 8 PT cells, PDSS-26 cells, RIT-3 cells, SCS214 cells, SN-SY-1 cells, STS255 cells, STSAR-198 cells, STSAR-84 cells, SW1045 cells, SYN-1 cells, SYNb-1 cells, SYNb-2 cells, YaFUSS cells, or any combination thereof.

In some cases, the method of reducing proliferation and/or inducing apoptosis and/or inducing necrosis can comprise treating the population of cells with a compound inhibiting the conjugation of a small ubiquitin-like modifier (SUMO). The method can comprise treating the population of cells with a compound inhibiting the conjugation of small ubiquitin-like modifier 1 (SUMO1). The method can comprise treating the population of cells with a compound inhibiting the conjugation of small ubiquitin-like modifier 2 (SUMO2). The method can comprise treating the population of cells with a compound inhibiting the conjugation of small ubiquitin-like modifier 3 (SUMO3). The method can comprise treating the population of cells with a compound inhibiting the conjugation of small ubiquitin-like modifier 4 (SUMO4).

In some aspects, the method of reducing proliferation and/or inducing apoptosis and/or inducing necrosis can comprise treating the population of cells with a SUMOylation inhibitor. In some cases, the method of reducing proliferation and/or inducing apoptosis and/or inducing necrosis can comprise treating the population of cells with a SUMO E1 inhibitor. In some cases, the method of reducing proliferation and/or inducing apoptosis and/or inducing necrosis can comprise treating the population of cells with a SUMOE1 inhibitor such as ginkgolic acid. In some cases, the method of reducing proliferation and/or inducing apoptosis and/or inducing necrosis can comprise treating the population of cells with a SUMO E1 inhibitor such as anacardic acid. In some cases, the method of reducing proliferation and/or inducing apoptosis and/or inducing necrosis can comprise treating the population of cells with a SUMO E1 inhibitor such as kerriamycin B. In some cases, the method of reducing proliferation and/or inducing apoptosis and/or inducing necrosis can comprise treating the population of cells with a SUMO E1 inhibitor such as davidiin. In some cases, the method of reducing proliferation and/or inducing apoptosis and/or inducing necrosis can comprise treating the population of cells with a SUMO E1 inhibitor such as CID9549553. In some cases, the method of reducing proliferation and/or inducing apoptosis and/or inducing necrosis can comprise treating the population of cells with a SUMO E1 inhibitor such as COH000. In some cases, the method of reducing proliferation and/or inducing apoptosis and/or inducing necrosis can comprise treating the population of cells with a SUMO E2 inhibitor. In some cases, the method of reducing proliferation and/or inducing apoptosis and/or inducing necrosis can comprise treating the population of cells with a SUMO E2 inhibitor such as Ubc9. In some cases, the method of reducing proliferation and/or inducing apoptosis and/or inducing necrosis can comprise treating the population of cells with a SUMO E2 inhibitor such as GSK145A. In some cases, the method of reducing proliferation and/or inducing apoptosis and/or inducing necrosis can comprise treating the population of cells with a SUMO E2 inhibitor such as Compound 1.

In some embodiments, the method can comprise administration of Compound 1 or a pharmaceutically acceptable salt or pharmaceutically acceptable composition thereof. In some aspects, Compound 1 can be a SUMOylation inhibitor. In some cases, Compound 1 can inhibit SUMO E1. In some cases, Compound 1 can inhibit SUMO E2. In some cases, Compound 1 can inhibit SUMO E3. In some cases, Compound 1 can inhibit SUMO2 Activating Enzyme 1 (SAE1). In some cases, Compound 1 can inhibit SUMO Activating Enzyme 2 (SAE2).

In some embodiments, the method of reducing proliferation and/or inducing apoptosis and/or inducing necrosis in a population of cancer cells can comprise treating the population with a compound that inhibits expression of a fusion protein. In some aspects, the fusion protein described herein can be indicative of cancer. In some aspects, the fusion protein described herein can be indicative of sarcoma. In some aspects, the fusion protein described herein can be indicative of the presence of synovial sarcoma cells. In some aspects, the fusion protein described herein can be indicative of the presence of soft tissue sarcoma cells. In some aspects, the fusion protein described herein can be indicative of the presence of liposarcoma cells. In some aspects, the fusion protein described herein can be indicative of the presence of angiosarcoma cells. In some aspects, the fusion protein described herein can be indicative of the presence of epithelioid sarcoma cells. In some aspects, the fusion protein described herein can be indicative of the presence of leiomyosarcoma cells. In some aspects, the fusion protein described herein can be indicative of the presence of synovial sarcoma cells. In some aspects, the fusion protein described herein can be indicative of the presence of gastrointestinal stromal cancer cells. In some aspects, the fusion protein described herein can be indicative of the presence of fibrosarcoma cells. In some aspects, the fusion protein described herein can be indicative of the presence of clear cell sarcoma cells. In some aspects, the fusion protein described herein can be indicative of the presence of rhabdomyosarcoma cells. In some aspects, the fusion protein described herein can be indicative of the presence of dermatofibrosarcoma cells. In some aspects, the fusion protein described herein can be indicative of the presence of undifferentiated pleomorphic sarcoma cells. In some aspects, the fusion protein described herein can be indicative of the presence of fibrous histiocytoma cells. In some aspects, the fusion protein described herein can be indicative of the presence of Ewing sarcoma cells. In some aspects, the fusion protein described herein can be indicative of the presence of fibromatosis cells. In some aspects, the fusion protein described herein can be indicative of the presence of ganglioneuroblastoma cells. In some aspects, the fusion protein described herein can be indicative of the presence of chondrosarcoma cells. In some aspects, the fusion protein described herein can be indicative of the presence of myxoid chondrosarcoma cells. In some aspects, the fusion protein described herein can be indicative of the presence of hermangioendothelioma cells. In some aspects, the fusion protein described herein can be indicative of the presence of hemangioma cells. In some aspects, the fusion protein described herein can be indicative of the presence of PEComa cells. In some aspects, the fusion protein described herein can be indicative of the presence of spindle cell sarcoma cells.

In some embodiments, the method of reducing proliferation and/or inducing apoptosis and/or inducing necrosis in a population of cancer cells can comprise treating the population with a compound that inhibits expression of an SS18-SSX fusion protein in the population of cells. In some cases, the compound can inhibit expression of an SS18-SSX1 fusion protein in the population of cells. In some cases, the compound can inhibit expression of an SS18-SSX2 fusion protein in the population of cells. In some cases, the compound can inhibit expression of an SS18-SSX3 fusion protein in the population of cells. In some cases, the compound can inhibit expression of an SS18-SSX4 fusion protein in the population of cells. In some cases, the compound can inhibit expression of an SS18-SSS fusion protein in the population of cells. In some cases, the compound can inhibit expression of an SS18-SSX6 fusion protein in the population of cells. In some cases, the compound can inhibit expression of an SS18-SSX7 fusion protein in the population of cells. In some cases, the compound can inhibit expression of an SS18-SSX8 fusion protein in the population of cells. In some cases, the compound can inhibit expression of an SS18-SSX9 fusion protein in the population of cells. In some cases, the compound can inhibit expression of an SS18-Xp11 fusion protein in the population of cells. In some cases, the compound described herein can be Compound 1.

The SS18-SSX fusion protein described herein can be a combination of any SSX domains. The SS18-SSX1 fusion protein described herein can comprise an SSX1 domain comprising SEQ ID NO:2. The SS18-SSX2 fusion protein described herein can comprise an SSX2 domain comprising SEQ ID NO:3. The SS18-SSX3 fusion protein described herein can comprise an SSX3 domain comprising SEQ ID NO:4. The SS18-SSX4 fusion protein described herein can comprise an SSX4 domain comprising SEQ ID NO:5.

In some embodiments, the method of reducing proliferation and/or inducing apoptosis and/or inducing necrosis in a population of cancer cells can comprise downregulating one or more genes in the population of cancer cells. In some cases, the methods described herein can comprise downregulating no genes. In some cases, the methods described herein can comprise downregulating 1 gene. In some cases, the methods described herein can comprise downregulating 2 genes. In some cases, the methods described herein can comprise downregulating 3 genes. In some cases, the methods described herein can comprise downregulating 4 genes. In some cases, the methods described herein can comprise downregulating 5 genes. In some cases, the methods described herein can comprise downregulating 6 genes. In some cases, the methods described herein can comprise downregulating 7 genes. In some cases, the methods described herein can comprise downregulating 8 genes. In some cases, the methods described herein can comprise downregulating 9 genes. In some cases, the methods described herein can comprise downregulating 10 genes. In some cases, the methods described herein can comprise downregulating 11 genes. In some cases, the methods described herein can comprise downregulating 12 genes. In some cases, the methods described herein can comprise downregulating 13 genes. In some cases, the methods described herein can comprise downregulating 14 genes. In some cases, the methods described herein can comprise downregulating 15 genes. In some cases, the methods described herein can comprise downregulating 16 genes. In some cases, the methods described herein can comprise downregulating 17 genes. In some cases, the methods described herein can comprise downregulating 18 genes. In some cases, the methods described herein can comprise downregulating 19 genes. In some cases, the methods described herein can comprise downregulating 20 genes. In some cases, the methods described herein can comprise downregulating more than 20 genes.

In some embodiments, the method of reducing proliferation and/or inducing apoptosis and/or inducing necrosis in a population of cancer cells can comprise downregulating one or more genes involved in cancer cell proliferation or cancer cell survival. In some aspects, the methods described herein can comprise downregulating CDX2. In some aspects, the methods described herein can comprise downregulating CBX5. In some aspects, the methods described herein can comprise downregulating SUZ12. In some aspects, the methods described herein can comprise downregulating CENPA. In some aspects, the methods described herein can comprise downregulating HOXA10. In some aspects, the methods described herein can comprise downregulating CENPF. In some aspects, the methods described herein can comprise downregulating TYMS. In some aspects, the methods described herein can comprise downregulating AURKB. In some aspects, the methods described herein can comprise downregulating UBEC2C. In some aspects, the methods described herein can comprise downregulating HOXC10. In some aspects, the methods described herein can comprise downregulating SMC2. In some aspects, the methods described herein can comprise downregulating BCL2L11. In some aspects, the methods described herein can comprise downregulating ASPM. In some aspects, the methods described herein can comprise downregulating BUB1B. In some aspects, the methods described herein can comprise downregulating CENPA. In some aspects, the methods described herein can comprise downregulating NCAPG2. In some aspects, the methods described herein can comprise downregulating OIP5. In some aspects, the methods described herein can comprise downregulating PLK1. In some aspects, the methods described herein can comprise downregulating TOP2A. In some aspects, the methods described herein can comprise downregulating CDK1. In some aspects, the methods described herein can comprise downregulating HJURP. In some aspects, the methods described herein can comprise downregulating KIF4A. In some aspects, the methods described herein can comprise downregulating FANCI. In some aspects, the methods described herein can comprise downregulating one or more of CDX2, CBX5, SUZ12, CENPA, HOXA10, CENPF, TYMS, AURKB, UBE2C, BCL2L11, or any combination thereof. In some aspects, the methods described herein can comprise downregulating one or more of CDX2, CBX5, SUZ12, CENPA, HOXA10, CENPF, TYMS, AURKB, UBE2C, BCL2L11, ASPM, BUB1B, CENPA, NCAPG2, OIP5, PLK1, TOP2A, CDK1, HJURP, KIF4A, FANCI, or any combination thereof. In some aspects, the methods described herein can comprise downregulating one or more of CDX2, CBX5, SUZ12, CENPA, HOXA10, CENPF, TYMS, AURKB, UBE2C, HOXC10, SMC2, BCL2L11, or any combination thereof. In some aspects, the methods described herein can comprise downregulating one or more of CDX2, CBX5, SUZ12, CENPA, HOXA10, CENPF, TYMS, AURKB, UBE2C, HOXC10, SMC2, BCL2L11, ASPM, BUB1B, CENPA, NCAPG2, OIP5, PLK1, TOP2A, CDK1, HJURP, KIF4A, FANCI, or any combination thereof.

In some embodiments, the method of reducing proliferation and/or inducing apoptosis and/or inducing necrosis in a population of cancer cells can comprise upregulating one or more genes in the population of cancer cells. In some cases, the methods described herein can comprise upregulating no genes. In some cases, the methods described herein can comprise upregulating 1 gene. In some cases, the methods described herein can comprise upregulating 2 genes. In some cases, the methods described herein can comprise upregulating 3 genes. In some cases, the methods described herein can comprise upregulating 4 genes. In some cases, the methods described herein can comprise upregulating 5 genes. In some cases, the methods described herein can comprise upregulating 6 genes. In some cases, the methods described herein can comprise upregulating 7 genes. In some cases, the methods described herein can comprise upregulating 8 genes. In some cases, the methods described herein can comprise upregulating 9 genes. In some cases, the methods described herein can comprise upregulating 10 genes. In some cases, the methods described herein can comprise upregulating 11 genes. In some cases, the methods described herein can comprise upregulating 12 genes. In some cases, the methods described herein can comprise upregulating 13 genes. In some cases, the methods described herein can comprise upregulating 14 genes. In some cases, the methods described herein can comprise upregulating 15 genes. In some cases, the methods described herein can comprise upregulating 16 genes. In some cases, the methods described herein can comprise upregulating 17 genes. In some cases, the methods described herein can comprise upregulating 18 genes. In some cases, the methods described herein can comprise upregulating 19 genes. In some cases, the methods described herein can comprise upregulating 20 genes. In some cases, the methods described herein can comprise upregulating more than 20 genes.

In some embodiments, the method of reducing proliferation and/or inducing apoptosis and/or inducing necrosis in a population of cancer cells can comprise upregulating one or more genes involved in cancer cell proliferation or cancer cell survival. In some aspects, the methods described herein can comprise upregulating GADD45B. In some aspects, the methods described herein can comprise upregulating KLF4. In some aspects, the methods described herein can comprise upregulating NCOA3. In some aspects, the methods described herein can comprise upregulating CXCR4. In some aspects, the methods described herein can comprise upregulating GDF15. In some aspects, the methods described herein can comprise upregulating BCL2L11. In some aspects, the methods described herein can comprise upregulating HMMR. In some aspects, the methods described herein can comprise upregulating NEMP1. In some aspects, the methods described herein can comprise upregulating CDC20. In some aspects, the methods described herein can comprise upregulating AURKA. In some aspects, the methods described herein can comprise upregulating PRC1. In some aspects, the methods described herein can comprise upregulating MKI67. In some aspects, the methods described herein can comprise upregulating FOXM1. In some aspects, the methods described herein can comprise upregulating HMGB2. In some aspects, the methods described herein can comprise upregulating RRM1. In some aspects, the methods described herein can comprise upregulating RRM2. In some aspects, the methods described herein can comprise upregulating POLA2. In some aspects, the methods described herein can comprise upregulating NUSAP1. In some aspects, the methods described herein can comprise upregulating BIRC5. In some aspects, the methods described herein can comprise upregulating LMNB1. In some aspects, the methods described herein can comprise upregulating TPX2. In some aspects, the methods described herein can comprise upregulating ATAD2. In some aspects, the methods described herein can comprise upregulating BUB1. In some aspects, the methods described herein can comprise upregulating POLQ. In some aspects, the methods described herein can comprise upregulating RFC4. In some aspects, the methods described herein can comprise upregulating ZWINT. In some aspects, the methods described herein can comprise upregulating CDC6. In some aspects, the methods described herein can comprise upregulating CENPM. In some aspects, the methods described herein can comprise upregulating CDCA8. In some aspects, the methods described herein can comprise upregulating NCAPG. In some aspects, the methods described herein can comprise upregulating DSCC1. In some aspects, the methods described herein can comprise upregulating GINS1. In some aspects, the methods described herein can comprise upregulating MAD2L1. In some aspects, the methods described herein can comprise upregulating RAD51AP1. In some aspects, the methods described herein can comprise upregulating PLK4. In some aspects, the methods described herein can comprise upregulating PBK. In some aspects, the methods described herein can comprise upregulating one or more of GADD45B, KLF4, NCOA3, BCL2L11, or any combination thereof. In some aspects, the methods described herein can comprise upregulating one or more of GADD45B, KLF4, NCOA3, BCL2L11, HMMR, NEMP1, CDC20, AURKA, PRC1, MK167, FOXM1, HMGB2, RRM1, RRM2, POLA2, NUSAP1, BIRC5, LMNB1, TPX2, ATAD2, BUB1, POLQ, RFC4, ZWINT, CDC6, CENPM, CDCA8, NCAPG, DSCC1, GINS1, MAD2L1, RAD51AP1, PBK, or any combination thereof. In some aspects, the methods described herein can comprise upregulating one or more of GADD4SB, KLF4, NCOA3, CXCR4, GDF15, BCL2L11, or any combination thereof. In some aspects, the methods described herein can comprise upregulating one or more of GADD45B, KLF4, NCOA3, CXCR4, GDF15, BCL2L11, HMMR, NEMP1, CDC20, AURKA, PRC1, MK167, FOXM1, HMGB2, RRM1, RRM2, POLA2, NUSAP1, BIRC5, LMNB1, TPX2, ATAD2, BUB1, POLQ, RFC4, ZWINT, CDC6, CENPM, CDCA8, NCAPG, DSCC1, GINS1,MAD2L1, RAD51AP1, PBK, or any combination thereof.

### Dosage

In some embodiments, the method of treatment can comprise administering about between about Img to about 150 mg of the compound or pharmaceutically acceptable salt. In some cases, the method herein can comprise administering about Img of the compound or pharmaceutically acceptable salt. In some cases, the method herein can comprise administering about 1.5 mg of the compound or pharmaceutically acceptable salt. In some cases, the method herein can comprise administering about 2 mg of the compound or pharmaceutically acceptable salt. In some cases, the method herein can comprise administering about 2.5 mg of the compound or pharmaceutically acceptable salt. In some cases, the method herein can comprise administering about 3 mg of the compound or pharmaceutically acceptable salt. In some cases, the method herein can comprise administering about 4 mg of the compound or pharmaceutically acceptable salt. In some cases, the method herein can comprise administering about 5 mg of the compound or pharmaceutically acceptable salt. In some cases, the method herein can comprise administering about 6 mg of the compound or pharmaceutically acceptable salt. In some cases, the method herein can comprise administering about 7 mg of the compound or pharmaceutically acceptable salt. In some cases, the method herein can comprise administering about 8 mg of the compound or pharmaceutically acceptable salt. In some cases, the method herein can comprise administering about 9 mg of the compound or pharmaceutically acceptable salt. In some cases, the method herein can comprise administering about 10 mg of the compound or pharmaceutically acceptable salt.

In some cases, the method herein can comprise administering about 12 mg of the compound or pharmaceutically acceptable salt. In some cases, the method herein can comprise administering about 14 mg of the compound or pharmaceutically acceptable salt. In some cases, the method herein can comprise administering about 16 mg of the compound or pharmaceutically acceptable salt. In some cases, the method herein can comprise administering about 18 mg of the compound or pharmaceutically acceptable salt. In some cases, the method herein can comprise administering about 20 mg of the compound or pharmaceutically acceptable salt. In some cases, the method herein can comprise administering about 25 mg of the compound or pharmaceutically acceptable salt. In some cases, the method berein can comprise administering about 30 mg of the compound or pharmaceutically acceptable salt. In some cases, the method herein can comprise administering about 35 mg of the compound or pharmaceutically acceptable salt. In some cases, the method herein can comprise administering about 40 mg of the compound or pharmaceutically acceptable salt. In some cases, the method herein can comprise administering about 45 mg of the compound or pharmaceutically acceptable salt. In some cases, the method herein can comprise administering about 50 mg of the compound or pharmaceutically acceptable salt. In some cases, the method herein can comprise administering about 55 mg of the compound or pharmaceutically acceptable salt. In some cases, the method herein can comprise administering about 60 mg of the compound or pharmaceutically acceptable salt. In some cases, the method herein can comprise administering about 65 mg of the compound or pharmaceutically acceptable salt. In some cases, the method herein can comprise administering about 70 mg of the compound or pharmaceutically acceptable salt. In some cases, the method herein can comprise administering about 75 mg of the compound or pharmaceutically acceptable salt.

In some cases, the method herein can comprise administering about 80 mg of the compound or pharmaceutically acceptable salt. In some cases, the method herein can comprise administering about 90 mg of the compound or pharmaceutically acceptable salt. In some cases, the method herein can comprise administering about 100 mg of the compound or pharmaceutically acceptable salt. In some cases, the method herein can comprise administering about 110 mg of the compound or pharmaceutically acceptable salt. In some cases, the method herein can comprise administering about 120 mg of the compound or pharmaceutically acceptable salt. In some cases, the method herein can comprise administering about 130 mg of the compound or pharmaceutically acceptable salt. In some cases, the method herein can comprise administering about 140 mg of the compound or pharmaceutically acceptable salt. In some cases, the method herein can comprise administering about 150 mg of the compound or pharmaceutically acceptable salt. In some cases, the method herein can comprise administering more than about 150 mg of the compound or pharmaceutically acceptable salt.

### Subjects

In some aspects, the subject is a mammal. In some aspects, the subject is a human. In some aspects, the subject is a human patient. In some aspects, the subject can have or is suspected of having a disorder or health condition such as a cancer. In some aspects, the subject can have or is suspected of having a sarcoma. In some aspects, the subject can have or is suspected of having a soft tissue sarcoma. In some aspects, the subject can have or is suspected of having liposarcoma. In some aspects, the subject can have or is suspected of having angiosarcoma. In some aspects, the subject can have or is suspected of having epithelioid sarcoma. In some aspects, the subject can have or is suspected of having leiomyosarcoma. In some aspects, the subject can have or is suspected of having synovial sarcoma. In some aspects, the subject can have or is suspected of having gastrointestinal stromal sarcoma. In some aspects, the subject can have or is suspected of having fibrosarcoma. In some aspects, the subject can have or is suspected of having clear cell sarcoma. In some aspects, the subject can have or is suspected of having rhabdomyosarcoma. In some aspects, the subject can have or is suspected of having dermatofibrosarcoma. In some aspects, the subject can have or is suspected of having undifferentiated pleomorphic sarcoma. In some aspects, the subject can have or is suspected of having fibrous histiocytomas. In some aspects, the subject can have or is suspected of having Ewing sarcoma. In some aspects, the subject can have or is suspected of having fibromatosis. In some aspects, the subject can have or is suspected of having ganglioneuroblastoma. In some aspects, the subject can have or is suspected of having chondrosarcoma. In some aspects, the subject can have or is suspected of having myxoid chondrosarcoma. In some aspects, the subject can have or is suspected of having hemangioendothelioma. In some aspects, the subject can have or is suspected of having hemangioma. In some aspects, the subject can have or is suspected of having PEComa. In some aspects, the subject can have or is suspected of having spindle cell sarcoma. In some aspects, the subject is healthy. In some cases, the subject can have or is suspected of having cancer of the extremities. In some aspects, the subject can have soft tissue sarcoma. In some aspects, the subject can have soft tissue sarcoma of the extremities. In some aspects, the subject can have synovial sarcoma. In some aspects, the subject can have synovial sarcoma of the extremities. In some aspects, the subject can have or be suspected of having a disease, condition, or disorder as disclosed herein.

The subject can be male. The subject can be female. The subject can be intersex. The gender of the subject can be determined according to the definitions of the American Medical Association (AMA). The subject can be a neonate, infant, child, adolescent, adult or senior according to the definitions of the American Medical Association (AMA). The subject can be a pediatric subject or an adult subject. In some embodiments, the subject is less than 20 years of age, less than 19 years of age, less than 18 years of age, less than 17 years of age, less than 16 years of age, less than 15 years of age, or less than 14 years of age, less than 13 years of age, less than 12 years of age, less than 11 years of age, less than 10 years of age, less than 9 years of age, less than 8 years of age, less than 7 years of age, less than 6 years of age, less than 5 years of age, less than 4 years of age, less than 3 years of age, less than 2 years of age, less than 1 year of age, or less than 1 month of age, or an age within a range defined by any of the preceding values. In some embodiments, the subject is less than 18 years of age. In some embodiments, the subject is less than 17 years of age. In some embodiments, the subject is less than 16 years of age. In some embodiments, the subject is less than 15 years of age. In some embodiments, the subject is less than 14 years of age. In some embodiments, the subject is less than 13 years of age. In some embodiments, the subject is less than 12 years of age. In some embodiments, the subject is less than 11 years of age. In some embodiments, the subject is less than 10 years of age. In some embodiments, the subject is less than 9 years of age. In some embodiments, the subject is less than 8 years of age. In some embodiments, the subject is less than 7 years of age. In some embodiments, the subject is less than 6 years of age. In some embodiments, the subject is less than 5 years of age. In some embodiments, the subject is less than 4 years of age. In some embodiments, the subject is less than 3 years of age. In some embodiments, the subject is less than 2 years of age. In some embodiments, the subject is less than 1 year of age. In some embodiments, the subject is less than 12 months of age. In some embodiments, the subject is less than 11 months of age. In some embodiments, the subject is less than 10 months of age. In some embodiments, the subject is less than 9 months of age. In some embodiments, the subject is less than 8 months of age. In some embodiments, the subject is less than 7 months of age. In some embodiments, the subject is less than 6 months of age. In some embodiments, the subject is less than 5 months of age. In some embodiments, the subject is less than 4 months of age. In some embodiments, the subject is less than 3 months of age. In some embodiments, the subject is less than 2 months of age. In some embodiments, the subject is less than 1 month of age.

### Biomarkers

In some embodiments, the concentration, presence, or absence, or any combination thereof, of a biomarker can be measured. In some cases, the biomarker can be indicative of a genetic modification. In some cases, the biomarker can be measured utilizing clinical procedures comprising tissue biopsy, blood draw, liquid biopsy, or combinations thereof. In some cases, the biomarker can be measured utilizing molecular biology techniques comprising qPCR, qRT-PCR, ChIP-Seq, Immunoblots, Cut&Run, Next-Gen sequencing, DNA microarrays, or combinations thereof. In some cases, the biomarker can be indicative of a genetic modification. In some cases, the biomarker can be indicative of knockdown of a genetic target. In some cases, the biomarker can be indicative of upregulation or downregulation of a protein. In some cases, the biomarker can be indicative of increased or decreased proliferation in a population of cells. In some cases, the biomarker can be a protein. In some cases, the biomarker can be H2AK119Ub. In some cases, the biomarker can be the SS18-SSX fusion protein. In some cases, the biomarker can be Ki-67.

In some cases, the methods disclosed herein can include expression of H2AK119Ub in a population of cancer cells in a subject. In some cases, the methods disclosed herein can include expression of SS18-SSX in a population of cancer cells in a subject. In some cases, the methods disclosed herein can include expression of H2AK119ub in a population of cancer cells in a subject. In some cases, the method can comprise administering to the subject a small ubiquitin-like modifier 2 (SUMO-2) enzyme inhibitor. In some cases, the SUMO-2 enzyme inhibitor can inhibit SUMO2-activating enzyme 1 (SAE1). In some cases, the SUMO-2 enzyme inhibitor can inhibit SUMO2-activating enzyme 2 (SAE2). In some cases, population of cancer cells can comprise a population of synovial sarcoma cells. In some cases, the population of synovial sarcoma cells can comprise a population of cell line 1273/99 cells. In some cases, the population of synovial sarcoma cells can comprise a population of YAMATO-SS cells. In some cases, the population of cells can comprise a population of HS-SY-II cells. In some case, the population of cells can comprise a population of Aska-SS cells. In some cases, the population can comprise a population of SYO1 cells.

In some embodiments, the method can comprise inducing a downregulation of one or more genes in the population of cancer cells. In some cases, the method can comprise inducing a downregulation of at least one, two, three, four, five, six, seven, eight, nine, ten, eleven, twelve, thirteen, fourteen, fifteen, sixteen, seventeen, eighteen, nineteen, twenty, or more than twenty genes in the population of cancer cells. In some cases, the SUMO-2 enzyme inhibitor can be Compound 1, or pharmaceutically acceptable salt thereof.

In some embodiments, H2AK119Ub concentration can be measured. In some cases, H2AK119Ub downregulation can be measured. In some cases, H2AK119Ub downregulation can be measured utilizing clinical procedures comprising tissue biopsy, blood draw, liquid biopsy, or combinations thereof. In some cases, H2AK119Ub can be measured utilizing molecular biology techniques comprising qPCR, qRT-PCR, ChIP-Seq, Immunoblots, Cut&Run, Next-Gen sequencing, DNA microarrays, or combinations thereof. In some cases, H2AK119Ub downregulation can be measured as an indication of downregulation of another gene or protein. In some cases, H2AK119Ub downregulation can be indicative of decreased proliferation in a population of cells. In some cases, H2AK119Ub concentration or downregulation can be measured as an indication of SS18-SSX fusion protein downregulation. In some embodiments, H2AK119Ub concentration or downregulation can be measured as an indication of effectiveness of a treatment. In some cases, H2AK119Ub downregulation or concentration can be measured as an indication of effectiveness of contact or treatment with Compound 1.

In some embodiments, H2AK119Ub chromatin occupancy at SySa direct target genes can be measured in a population of cells. In some cases, H2AK119Ub chromatin occupancy at SySa direct target genes can be measured utilizing clinical procedures comprising tissue biopsy, blood draw, liquid biopsy, or combinations thereof. In some cases, H2AK119Ub chromatin occupancy at SySa direct target genes can be measured utilizing molecular biology techniques comprising qPCR, qRT-PCR, ChIP-Seq, Immunoblots, Cut&Run, Next-Gen sequencing, DNA microarrays, or combinations thereof. In some cases, reduction of H2AK119Ub chromatin occupancy at SySa direct target genes can be measured as an indication of downregulation of another gene or protein. In some cases, H2AK119Ub downregulation can be indicative of decreased proliferation in a population of cells. In some cases, reduction of H2AK119Ub chromatin occupancy at SySa direct target genes can be measured as an indication of SS18-SSX fusion protein downregulation. In some embodiments, reduction of H2AK119Ub chromatin occupancy at SySa direct target genes can be measured as an indication of effectiveness of a treatment. In some cases, reduction of H2AK119Ub chromatin occupancy at SySa direct target genes can be measured as an indication of effectiveness of contact or treatment with Compound 1. In some embodiments, SySa direct target genes include genes that are stereotypically up or downregulated in patients with SySa due to SS18-SSX fusion protein activity. In some cases, SySa direct target genes comprise CDX2, CBX5, SUZ12, CENPA, HOXA10, CENPF, TYMS, AURKB, UBE2C, BCL2L11, HOXC10, SMC2, GADD45B, KLF4, NCOA3, CXCR4, GDF15, or combinations thereof.

In some embodiments, SS18-SSX fusion protein concentration or downregulation can be measured in a population of cells. In some cases, SS18-SSX fusion protein concentration or downregulation can be measured utilizing clinical procedures comprising tissue biopsy, blood draw, liquid biopsy, or combinations thereof. In some cases, SS18-SSX fusion protein concentration or downregulation can be measured utilizing molecular biology techniques comprising qPCR, qRT-PCR, ChIP-Seq, Immunoblots, Cut&Run, Next-Gen sequencing, DNA microarrays, or combinations thereof. In some cases, SS18-SSX fusion protein concentration or downregulation can be measured as an indication of downregulation of another gene or protein. In some cases, SS18-SSX fusion protein concentration or downregulation can be indicative of decreased proliferation in a population of cells. In some cases, SS18-SSX fusion protein concentration or downregulation can be measured as an indication of SUMO2 downregulation. In some embodiments, SS18-SSX fusion protein concentration or downregulation can be measured as an indication of effectiveness of a treatment. In some cases, SS18-SSX fusion protein concentration or downregulation can be measured as an indication of effectiveness of contact or treatment with Compound 1.

In some embodiments, SS18-SSX fusion protein chromatin occupancy at SySa direct target genes in a population of cells can be measured. In some cases, SS18-SSX fusion protein occupancy at SySa direct target genes can be measured utilizing clinical procedures comprising tissue biopsy, blood draw, liquid biopsy, or combinations thereof. In some cases, SS18-SSX fusion protein occupancy at SySa direct target genes can be measured utilizing molecular biology techniques comprising qPCR, qRT-PCR, ChIP-Seq, Immunoblots, Cut&Run, Next-Gen sequencing, DNA microarrays, or combinations thereof. In some cases, reduction of SS18-SSX fusion protein chromatin occupancy at SySa direct target genes can be measured as an indication of downregulation of another gene or protein. In some cases, reduction of SS18-SSX fusion protein chromatin occupancy at SySa direct target genes can be indicative of decreased proliferation in a population of cells. In some cases, reduction of SS18-SSX fusion protein chromatin occupancy at SySa direct target genes can be measured as an indication of SUMO-2 downregulation. In some embodiments, reduction of SS18-SSX fusion protein chromatin occupancy at SySa direct target genes can be measured as an indication of effectiveness of a treatment. In some cases, reduction of SS18-SSX fusion protein chromatin occupancy at SySa direct target genes can be measured as an indication of effectiveness of contact or treatment with Compound 1. In some embodiments, Synovial sarcoma direct target genes include genes that are stereotypically up or downregulated in SySa cells due to SS18-SSX fusion protein activity. In some embodiments, SySa direct target genes comprise CDX2, CBX5, SUZ12, CENPA, HOXA10, CENPF, TYMS, AURKB, UBE2C, BCL2L11, HOXC10, SMC2, GADD45B, KLF4, NCOA3, CXCR4, GDF15, or combinations thereof.

In some embodiments, Ki-67 concentration, or downregulation in a population of cells can be measured in a population of cells. In some cases, Ki-67 concentration or downregulation can be measured utilizing clinical procedures comprising tissue biopsy, blood draw, liquid biopsy, or combinations thereof. In some cases, Ki-67 concentration or downregulation can be measured utilizing molecular biology techniques comprising qPCR, qRT-PCR, ChIP-Seq, Immunoblots, Cut&Run, Next-Gen sequencing, DNA microarrays, or combinations thereof. In some cases, Ki-67 concentration or downregulation can be measured as an indication of downregulation of another gene or protein. In some cases, Ki-67 concentration or downregulation can be measured as an indication of decreased proliferation. In some embodiments, Ki-67 concentration or downregulation can be measured as an indication of effectiveness of a treatment. In some cases, Ki-67 concentration or downregulation can be measured as an indication of effectiveness of contact or treatment with Compound 1.

SEQ ID Table

| | | Protein | |
|---|---|---|---|
| | SEQ | Accession | |
| Name | ID | Number | Sequence |
| SS18 | | Q15532.3 | MSVAFAAPRQRGKGEITPAAIQKMLDDNNH |
| | | | LIQCIMDSQNKGKTSECSQYQQMLHTNLVY |
| | | | LATIADSNQNMQSLLPAPPTQNMPMGPGGM |
| | | | NQSGPPPPPRSHNMPSDGMVGGGPPAPHMQ |
| | | | NQMNGQMPGPNHMPMQGPGPNQLNMTNSSM |
| | | | NMPSSSHGSM GGYNHSVPSS QSMPVQNQMT |
| | | | MSQGQPMGNY GPRPNMSMQP NQGPMMHQQP |
| | | | PSQQYNMPQG GGQHYQGQQP PMGMMGQVNQ |
| | | | GNHMMGQRQI PPYRPPQQGP PQQYSGQEDY |
| | | | YGDQYSHGGQGPPEGMNQQY YPDGHNDYGY |
| | | | QQPSYPEQGY DRPYEDSSQH YYEGGNSQYG |
| | | | QQQDAYQGPP PQQGYPPQQQQYPG |
| | | | QQGYPGQQQGYGPSQGGPGPQYPNYPQGQG |
| | | | QQYGGYRPTQPGPPQP PQQRPYGYDQ |
| | | | GQYGNYQQ |
| SSX1 | 2 | Q16384.2 | MNGDDTFAKR PRDDAKASEK RSKAFDDIAT |
| | | | YFSKKEWKKM KYSEKISYVY MKRNYKAMTK |
| | | | LGFKVTLPPF MCNKQATDFQ GNDFDNDHNR |
| | | | RIQVEHPQMT FGRLHRIIPK IMPKKPAEDE |
| | | | NDSKGVSEAS GPQNDGKQLH PPGKANISEK |
| | | | INKRSGPKRG KHAWTHRLRE |
| | | | RKQL VIYEEISDPEEDDE |
| SSX2 | 3 | Q16385.2 | MNGDDAFARR PTVGAQIPEK IQKAFDDIAK |
| | | | YFSKEEWEKM KASEKIFYVY MKRKYEAMTK |
| | | | LGFKATLPPF MCNKRAEDFQ GNDLDNDPNR |
| | | | GNQVERPQMT FGRLQGISPK IMPKKPAEEG |
| | | | NDSEEVPEAS GPQNDGKELC PPGKPTTSEK |
| | | | IHERSGPKRG EHAWTHRLRE |
| | | | RKQLVIYEEISDPEEDDE |
| SSX3 | 4 | Q99909.2 | MNGDDTFARR PTVGAQIPEK IQKAFDDIAK |
| | | | YFSKEEWEKM KVSEKIVYVY MKRKYEAMTK |
| | | | LGFKAILPSF MRNKRVTDFQ GNDFDNDPNR |
| | | | GNQVQRPQMT FGRLQGIFPK IMPKKPAEEG |
| | | | NVSKEVPEAS GPQNDGKQLC PPGKPTTSEK |
| | | | INMISGPKRG EHAWTHRLRE |
| | | | RKQLVIYEEISDPEEDDE |
| SSX4 | 5 | O60224.1 | MNGDDAFARR PRDDAQISEK LRKAFDDIAK |
| | | | YFSKKEWEKM KSSEKIVYVY MKLNYEVMTK |
| | | | LGFKVTLPPF MRSKRAADFH GNDFGNDRNH |
| | | | RNQVERPQMT FGSLQRIFPK IMPKKPAEEE |
| | | | NGLKEVPEAS GPQNDGKQLC PPGNPSTLEK |
| | | | INKTSGPKRG KHAWTHRLRE |
| | | | RKQLVVYEEISDPEEDDE |

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Computational Analysis for Functional Genomic Screening

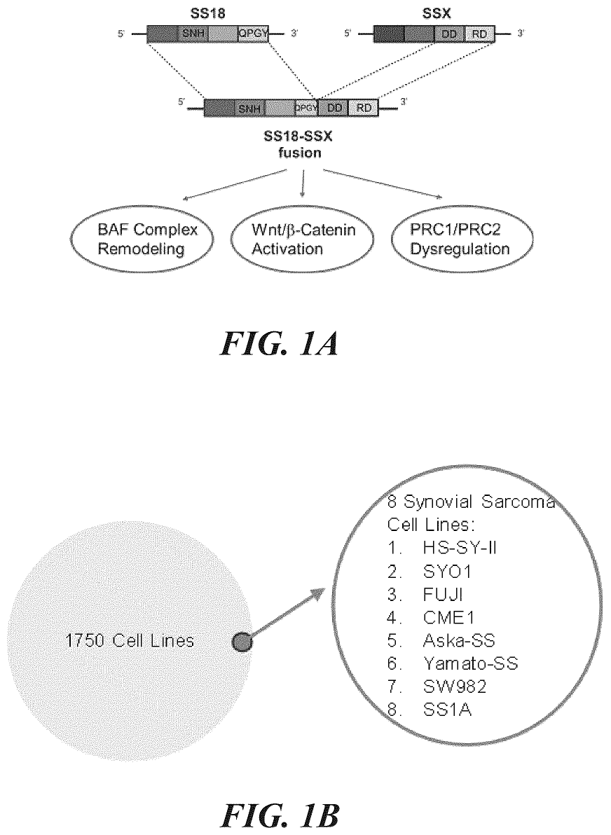

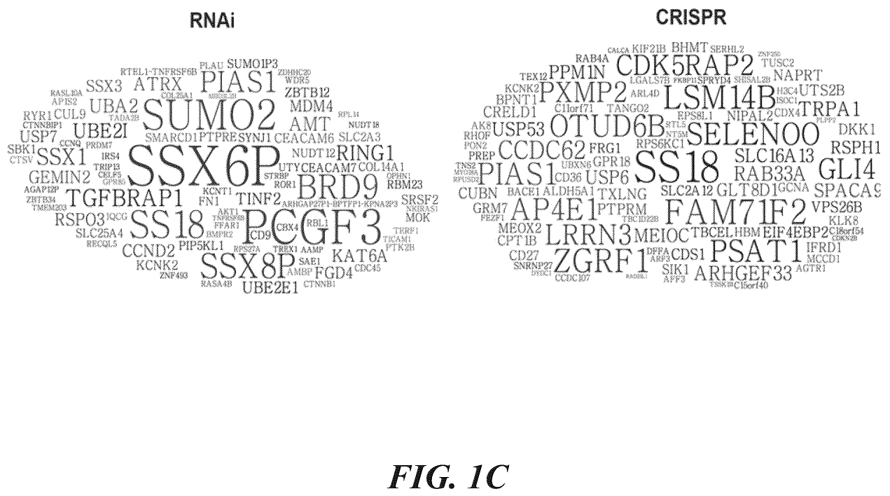

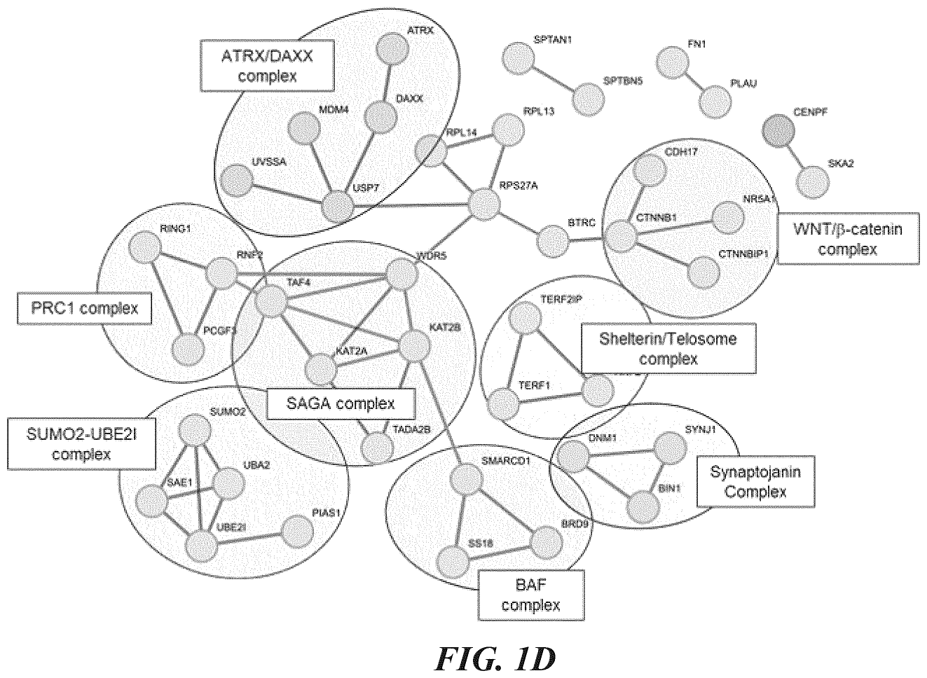

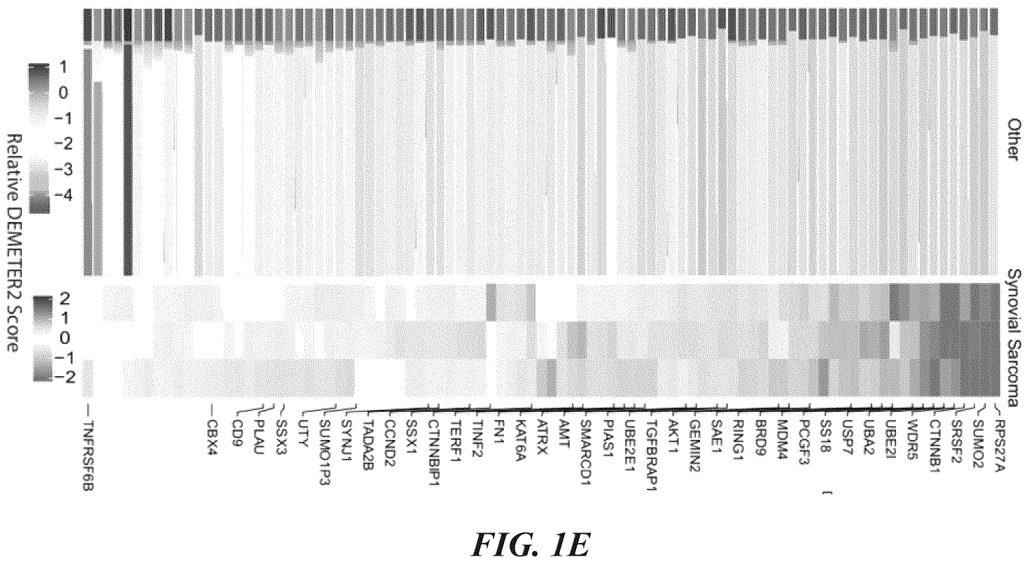

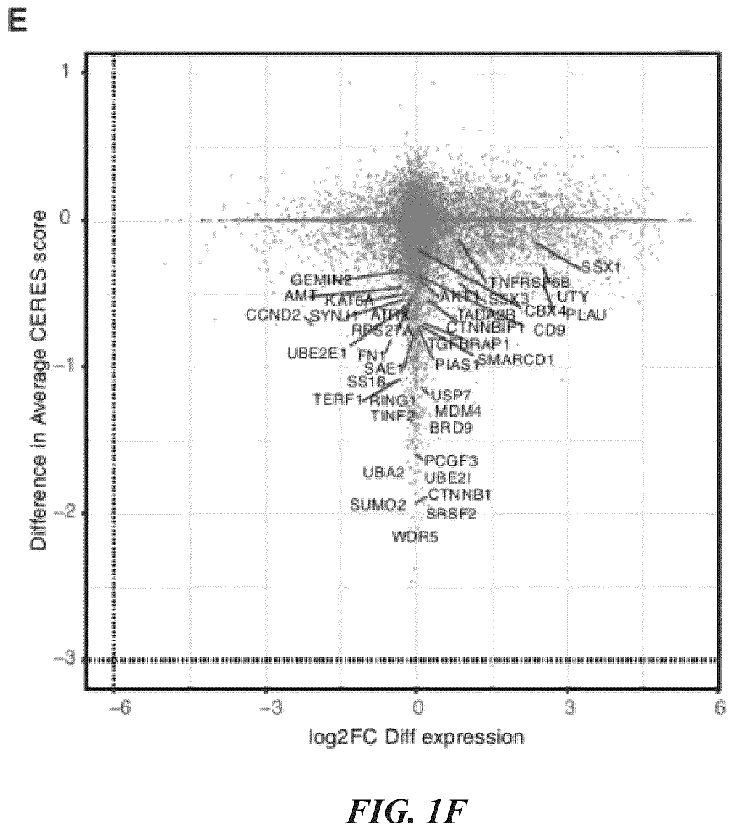

In order to investigate potential target genes of interest for CRISPR-Cas9 screening, the DepMap CRISPR-Cas9 database and RNAi database were analyzed. Potential target genes of interest predicted to be most relevant to synovial sarcoma cell functionality and proliferation were selected. Gene presence in the SS18-SSX fusion complex formation and functional characteristics were accounted for, such as BAF Complex Remodeling, Writ/β-Catenin Activation, and PRC1/PRC2 Dysregulation (FIG. 1A). From the 1750 possible cell lines contained in the DepMap CRISPR-Cas9 database, eight potential synovial sarcoma cell lines were selected for use in further screening (FIG. 1B). For each of the selected cell lines, the top 200 scoring genes for Chronos gene fitness, and the top 200 scoring genes for DEMETER2 RNAi gene dependency were selected and mapped (FIG. 1C). A complexing map was produced for relevant screening target genes, including mapping genes involved in formation of, for example, the WNT/β-Catenin complex, BAF complex, PRC1 complex, SUMO2-UBE2I complex, SAGA complex, Synaptojanin complex, Shelterin/Telesome complex, and ATRX/DAXX complex. (FIG. 1D)

Biochemical pathway analysis was conducted using Enrichr25 to identify potential enrichment for biological pathways in the SySa-selective dataset. This analysis revealed that there was a striking enrichment for the SUMO conjugation and SUMO transfer Reactome pathway and multiple members of the sumoylation machinery appeared as hits in the SySa-selective dependencies dataset including UBA2, SAE1, UBE2I, SUMO2 and, PIAS. (FIG. 1E) The fold change for each of these genes between SySa and non-SySa cancer cell lines in the Cancer Cell Line Encyclopedia (CCLE) and plotted it against the relative dependency values (FIG. 1F). Genes such as SSX1 and SSX3 were indeed much more highly expressed in SySa compared to non-SySa cell lines, genes such as BRD9, PCGF3, and SUMO2 had no noticeable difference in expression between these cell lines. This suggests that while the relatively higher dependency of SySa cell lines on genes downstream of the SS18-SSX fusion protein may result from their higher expression in cell lines from this lineage compared to others, the dependence on genes such BRD9, PCGF3, and SUMO2 may instead be explained by a relatively higher activity of these proteins in SySa compared to other cancers.

### Example 2: sgRNA Library Construction and Transfection for Genomic Screening

sgRNA Library Design

About 10 sgRNAs for each gene were designed utilizing GUIDES (Graphical User Interface for DNA Editing Screens). To design the sgRNAs, a variety of factors were integrated, including issue-specific exon expression from the GTEx Consortium, on-target activity scores, and strategies for off-target minimization, as well as functional protein domains based on Pfam data. The library included a percentage of about 5% of non-targeting guides, for a total library size of about 3665 guides. The sgRNAs for target screening genes of interest were designed using CRISPick.

sgRNA Library Synthesis

sgRNA libraries were synthesized using Array technology (CustomArray, Inc.), the library containing about 3310 guides targeting about 310 target genes of interest, along with about 174 guides included as non-targeting controls. The guides were amplified by PCR and cloned into pKLO.1 vector via ligation using the Esp3I(NEB) restriction sites. Transformations were performed using Invitrogen's MegaX DH10B TI electro-competent cells and utilizing an Eppendorf electroporator 2510 and Bio-Rad Gene Pulser 1 mm cuvettes. A minimum of about 30 million successfully transformed cells or about 30,000× coverage of the library was obtained.

sgRNA Transfection for Gene Editing

HEK-293T and SYO1 cells were cultured in DMEM supplemented with 10% FBS, 1% penicillin-streptomycin and 1% L-glutamine. HS-SY-II cells were additionally supplemented with 0.5% Sod. Pyruvate. HS-SY-II cells and SYO1 cells were authenticated by STR profiling. HEK-293T and SYO1 cells were cultured in DMEM supplemented with 10% FBS, 1% penicillin-streptomycin and 1% L-glutamine.

Lentivirus was produced in cultured HEK293T cells. Cells from about four 80% confluent 10 cm Petri dishes were transfected with about 0.9 μg of VSV-G envelope-expressing plasmid pMD2, about 9 μg of psPAX2 packaging vectors, and about 9 μg of the sgRNA library in the presence of 113.4 μL Polyethyleneimine (VWR International, 1 mg/mL) per plate. Growth media was exchanged after overnight incubation for about 12-14 hours, and supernatant containing the lentiviral pooled sgRNA library was collected after about 3 days. The supernatant containing the lentiviral pooled sgRNA library was then passed through a 0.45 μm filter and concentrated by centrifuging at about 6000 g for about 2 hours at about 4° C. The supernatant was then discarded, and the resulting pellets were resuspended in about 1/1000^{th} volume of PBS and rotated at about 4° C. overnight for about 12-14 hours. The concentrated lentiviral pool was flash frozen in ethanol-dry ice bath and stored at about −80° C.

### Example 3: Target Gene Screening in Synovial Sarcoma Cell Lines

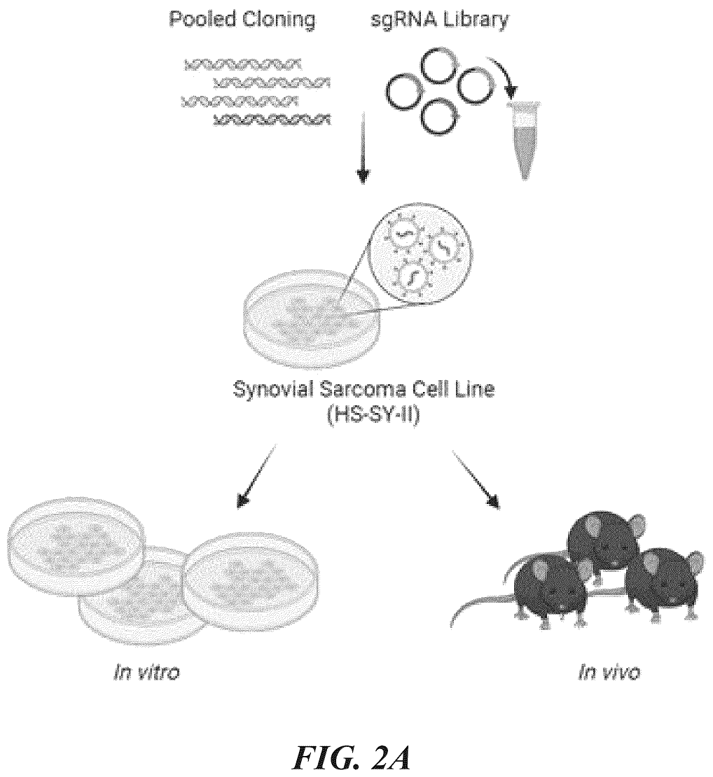

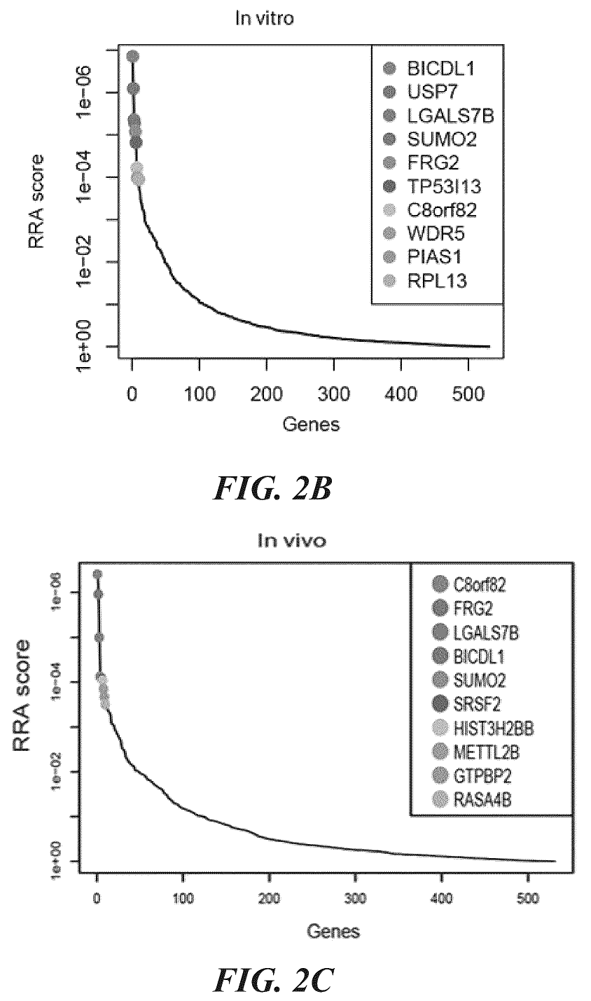

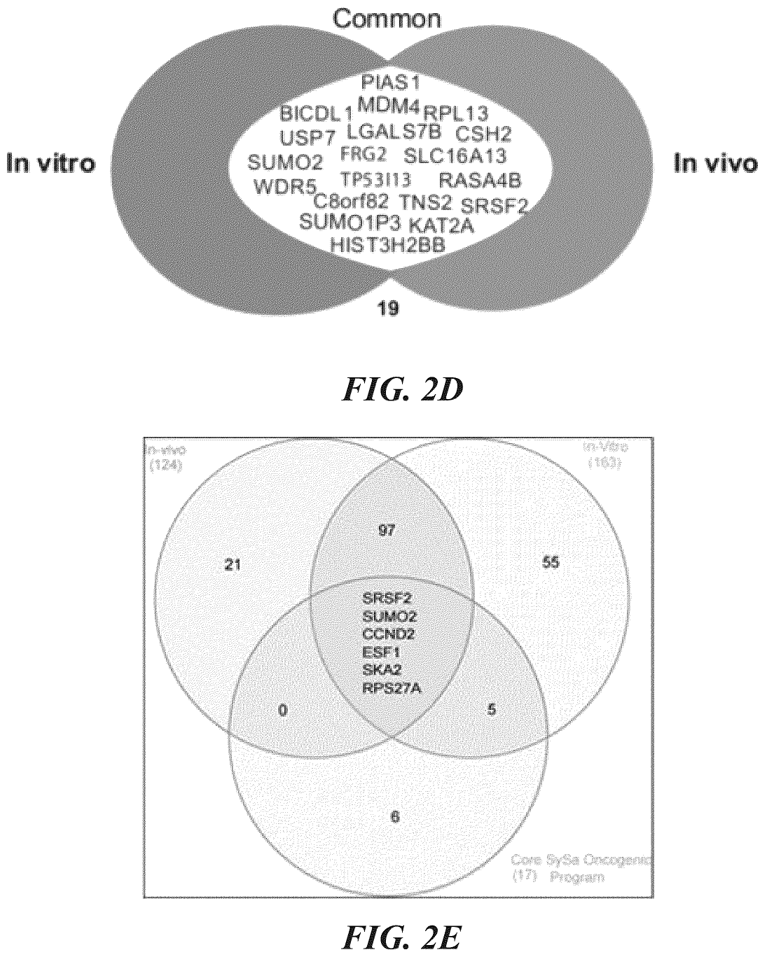

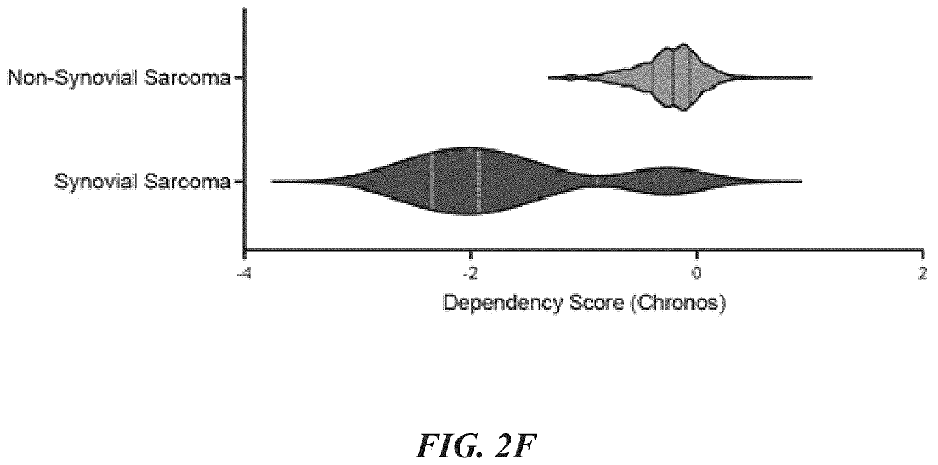

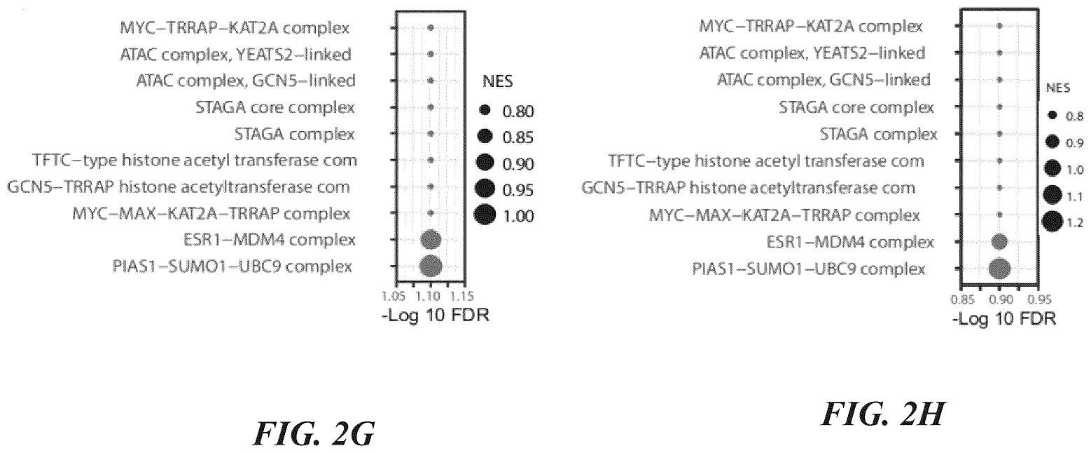

To evaluate the therapeutic potential of target candidate genes of interest, cellular activity, and cellular characteristics of CRISPR/Cas9 gene-edited HS-SY-II cells were evaluated prior to validation with additional cell lines. HS-SY-II cells were transduced with sgRNA from the pooled lentiviral sgRNA library targeting the AAVS site and cultured with puromycin for 2 days (FIG. 2A). The genomic DNA of CRISPR/Cas9 gene-edited HS-SY-II cells was sequenced, and the indel percentage was found to be about 92% with a knockout score of 90. Next, parallel in vivo and in vitro CRISPR screens were performed (FIG. 2A). HS-SY-II Cas9 cells were transduced with the pooled lentiviral sgRNA library in the presence of about 0.8 mg/mL of polybrene with an efficiency of about 30% or lower, to ensure most cells received a single sgRNA. After selection with puromycin (1 μg/mL) for about 2-4 days, a cell aliquot containing 5 million cells, about 1000× coverage of library, was frozen at about day 0. The remaining cells were divided into 2 arms for the in vivo and in vitro screens.

### In Vitro Screening

The HS-SY-II cells were cultured from each replicate for about 14 days (FIG. 2A). Samples were then obtained and sequenced via Illumina HiSeq. For the in vivo screen, 2 million cells in 50% Matrigel were transplanted subcutaneously into the flanks of 4 athymic nude mice per replicate. The resultant tumor was monitored, and mice were sacrificed when the tumor volume reached 1 cm³. The tumor was dissociated into single cell suspension using collagenase II (20 mg/mL) along with DNase I (10,000 Kunitz/mL) and used for further experiments. For the in vitro screens, at least 5 million cells were maintained throughout the 14-day culture period and collected at the end of the screen. Genomic DNA was extracted from collected cell pellets using a Zymo Quick DNA miniprep kit (#D3024) The sgRNA were PCR amplified by NEBNext High-Fidelity 2× PCR Master Mix (NEB) from the genomic DNA using the indexed PCR primers with next-generation sequencing adapters compatible with Illumina's NEXTERA kit. PCR products were size-selected by gel electrophoresis, quantified by Qubit (Thermo Fisher Scientific) and sequenced using HiSeq (Illumina).

### In Vivo Screening

In parallel, about 2 million cells (˜500× coverage) were subcutaneously injected in 50% Matrigel per replicate into the flanks of nude mice, with each replicate consisting of about 3 mice. In all, about 6 mice were injected, of which tumors developed in about 5. Mice were sacrificed and tumors were extracted at the end of 60 days. Samples were then obtained and sequenced via Illumina HiSeq.

Representation of sgRNAs in the replicates were analyzed. (FIG. 2B) sgRNA abundance and distribution for the target genes of interest were measured using MAGeCK Robust Rank Aggregation algorithm (FIG. 2B). Read counts for each gRNA were normalized. The MAGeCK and MAGeCK-VISPR algorithms were used to identify target genes where knockout leads to the loss of sgRNA when compared to non-targeted control genes. MAGeCK modules α-RRA (α-Robust Rank Aggregation) and MLE (maximum likelihood) were used to identify SgRNA that were selectively diminished in day 14 samples for the in vitro screen and tumor samples in the in vivo screen when compared to the starting sample (FIGS. 2B-2C). sgRNA read counts for each gene were compared at input reference (Day 0) and at the end of the screen to produce an RRA score in vitro and in vivo (FIG. 2C). Further screening of target genes of interest was performed via diagramming overlapping genes of interest with genes activated by the SS18-SSX oncoprotein (FIG. 2D). Chronos dependency score for small molecule inhibitors in synovial sarcoma cells and cells of other cancer types were measured to determine efficient targeting modalities. (FIG. 2E) Pathway enrichment analysis showed that the top hits were enriched for proteins involved in the SUMO complex in both in vitro as well as in vivo screens (FIGS. 2F-G)

### Example 4: Small Molecule Compound Screening

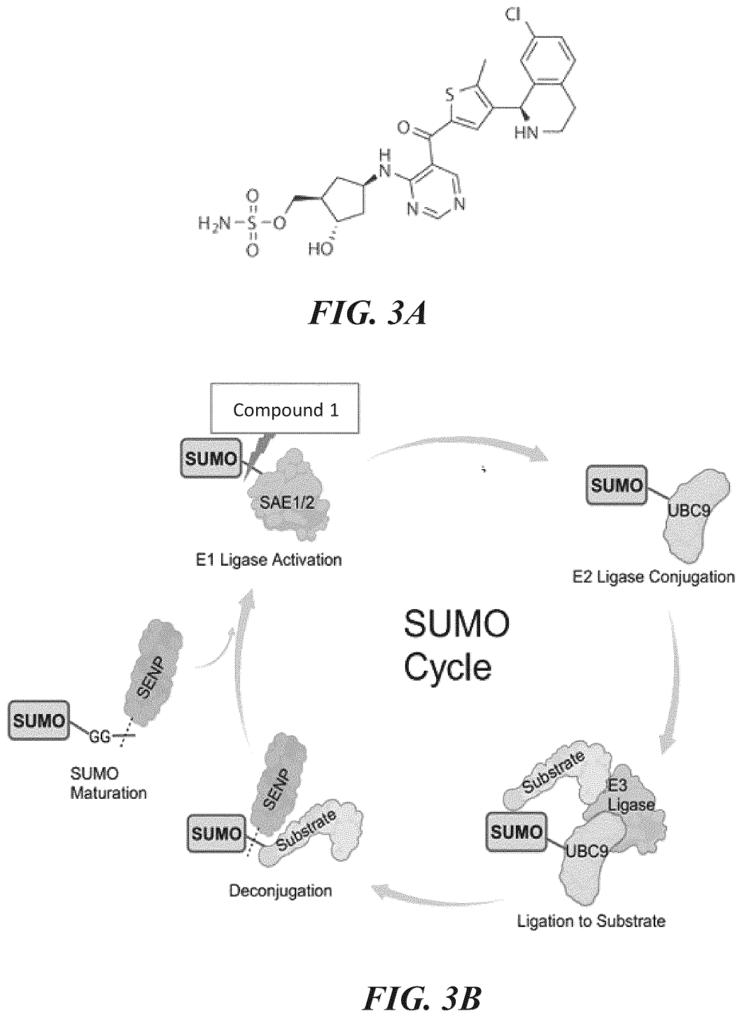

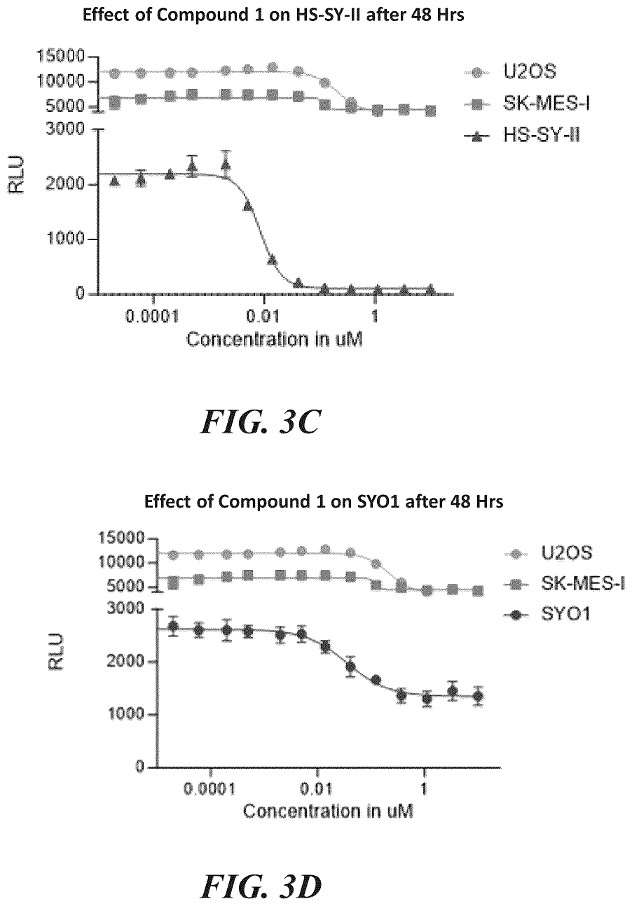

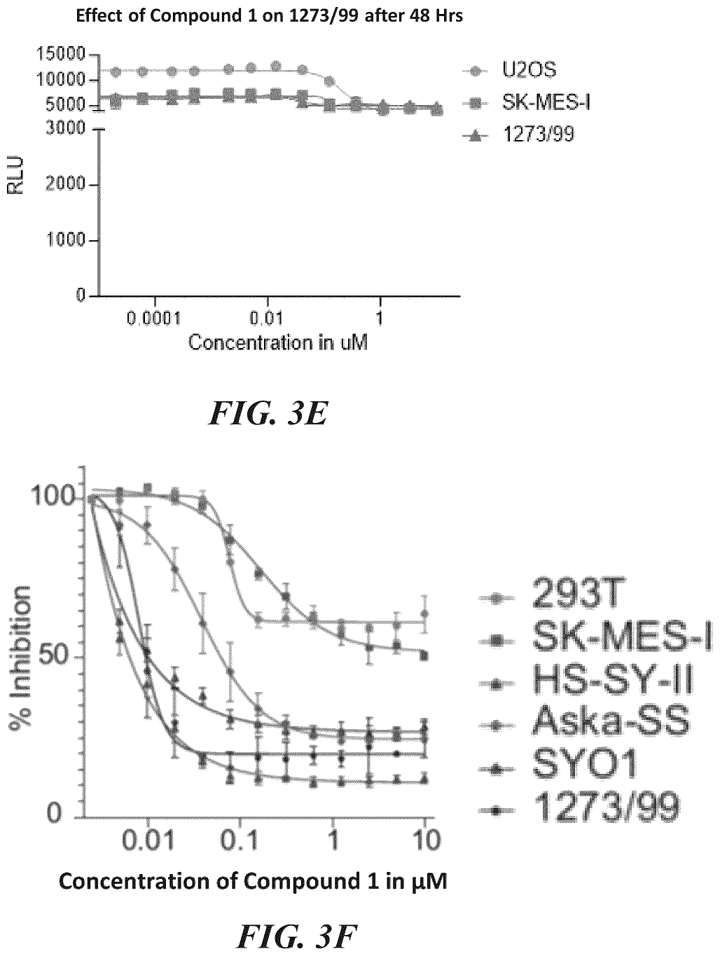

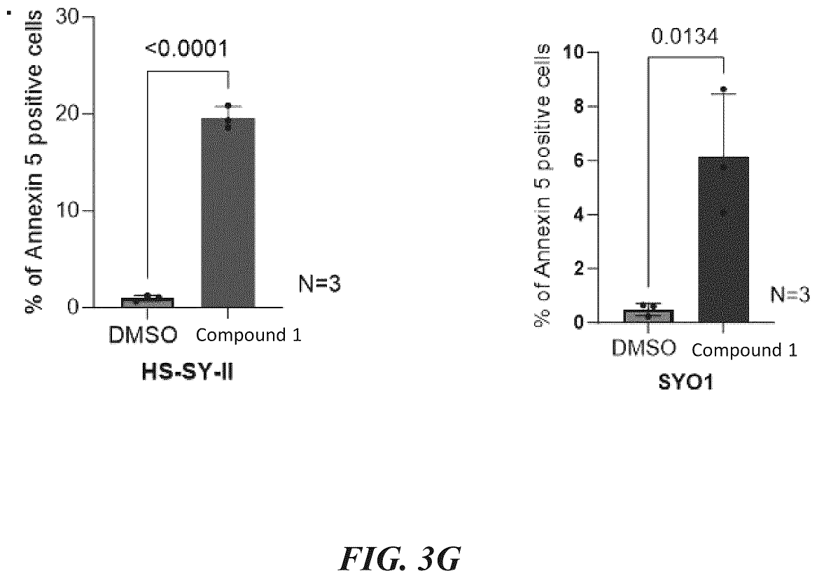

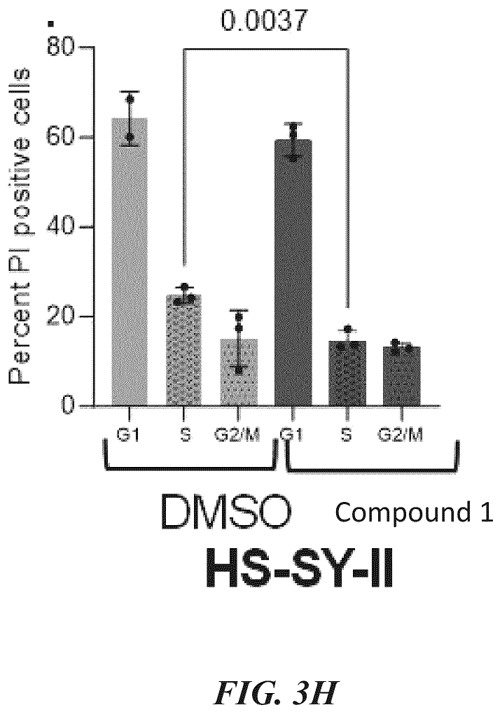

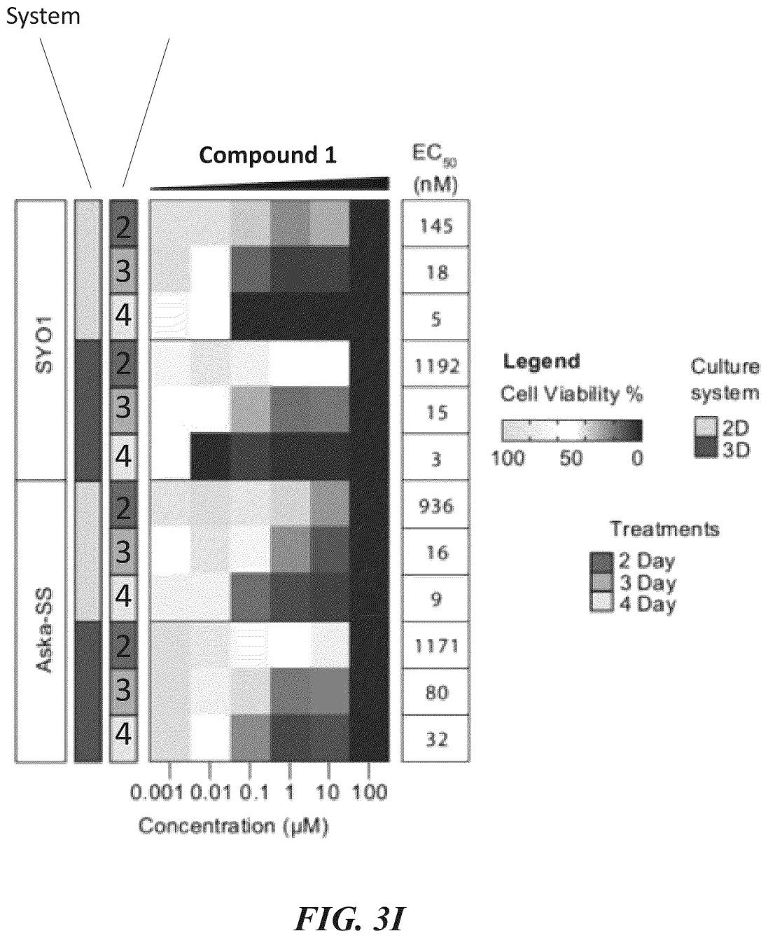

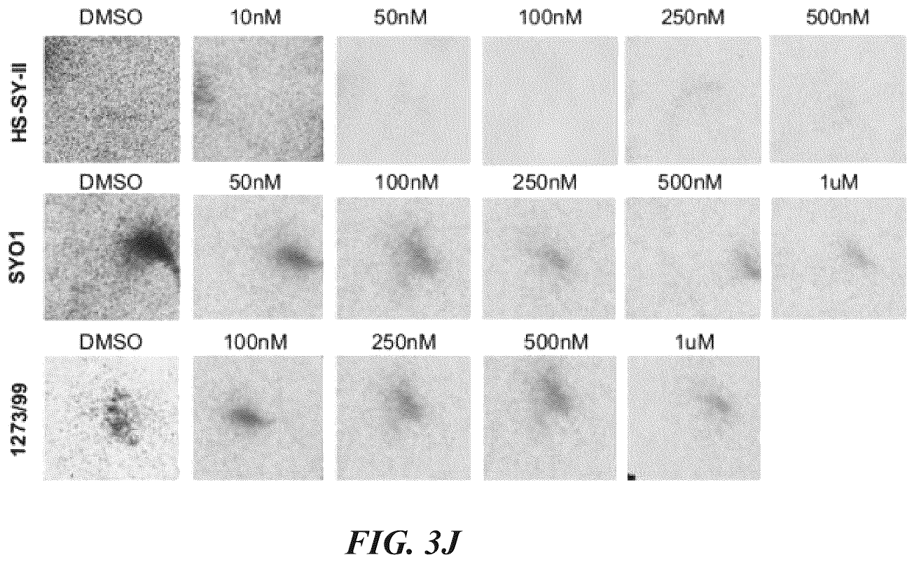

SUMO E1 inhibitor (Compound 1) was selected for screening (FIG. 3A). Compound 1 was found to interrupt the SUMO cycle at the E1 Ligase Activation step of Sumo-Activating Enzyme 1 (SAE1) or Sumo-Activating Enzyme 2 (SAE2) (FIG. 3B). Initial investigation into SAE inhibition by Compound 1 was performed at concentrations ranging from about 0 μM to about 1.5 μM. Cell viability was measured in RLU units for three different human synovial sarcoma cancer cell lines, SYO1, HS-SY-II, 1273/99, and an osteosarcoma cell line, U2OS, as well as a lung cancer cell line, SK-MESI (FIG. 3C-3E). Follow up investigations into SAE inhibition by Compound 1, at concentration ranges from 0 μM to 10 μM, were performed with the addition of the following cell lines: a control 293T line and the SySa line, Aska-SS. (FIG. 3F)

To investigate whether the reduction of proliferation by Compound 1 was due to apoptosis and necrosis in synovial sarcoma cell lines, flow cytometric analyses were performed with cells treated with Compound 1 and untreated cells stained with Annexin V-FITC apoptosis marker, as well as Propidium Iodide necrosis marker for cells in media containing Compound 1 and media only containing DMSO. The proliferation assay was performed using Cell Titer Glo Luminescent Cell Viability Assay (Promega). Cell numbers were optimized for 384-well plate for each cell line. SUMO2 inhibitors including Compound 1 were dissolved in DMSO and echo dotted onto a 384-well plate in varying concentrations, with the final concentration of DMSO at about 0.08% in each well. About 25 μL of cell suspension, a concentration of about 50,000 cells/mL were seeded in each well of a 384-well plate. The cells were incubated at about 37° C. at about 5% CO2 for about 48 hours, then quenched with CellTiter-Glo®, centrifuged at about 1000 rpm for about 1 minute, and incubated at about 20°° C. for about 20 min. Luminescence was recorded with a plate reader (BMG FLUOStar). The EC50 values were calculated by Graphpad Prism software. Apoptosis was quantified by flow cytometry using Annexin V-FITC kit from BD Biosciences. A concentration of about 3×10⁵ SYO1 and about 3×10⁵ HS-SY-II cells were seeded in a 6-well plate and allowed to attach for 24 hours. Compound 1 was added in varying concentrations and incubated for about 48 hours. After incubation, the cells were trypsinzed, washed in warm PBS, and resuspended in annexin V binding buffer. Annexin V-FITC was added and incubated at room temperature for about 10 minutes. The samples were then analyzed by flow cytometry using Fortessa along with FlowJo analysis software. Cell lines analyzed were HS-SY-II (left) and SYO1 (right) (FIG. 3G).

Cell cycle analysis was done by staining the cells with propidium iodide (PI). A concentration of about 3×10⁵ SYO1 and about 3×10⁵ HS-SY-II cells were seeded in a 6-well plate and allowed to attach for 24 hours. The cells were then exposed to varying concentrations of Compound 1 for 48 hours. Cells were trypsinzed, washed with PBS and fixed with ethanol. Cells were then washed and stained with PI. The samples were then analyzed by flow cytometry using Fortessa along with FlowJo analysis software. To measure cell proliferation via proportion of cells arrested in each phase of the cell cycle, percent PI of positive cells was measured for HS-SY-II cells in media containing Compound 1, as well as media containing only DMSO (FIG. 3H).

Further testing of Compound 1 was performed on 2D and 3D cultures for SYO1 and Aska-SS cell lines to determine whether these culture conditions resist Compound 1 treatment. In these studies, too, Compound 1 treatment led to a progressive and marked decrease in viability as measured by CellTiter-Glo over 2, 3, and 4 days (FIG. 3I). Colony-forming assays for the HS-SY-II (top), SYO1 (middle) and 12273/99 (bottom) cell lines using a Compound 1 titration series also demonstrated a dramatic and dose-dependent reduction in colony formation (FIG. 3J).

### Example 5: Target Genetic Screening for Knockdown Via Contact with Compound 1

To comprehensively interrogate the transcriptomic changes occurring in synovial sarcoma cells, HS-SY-II and SYO1 cells were cultured in media containing DMSO and media containing Compound 1 and subjected to total RNA sequencing. HS-SY-II and SYO1 cells were contacted with either Compound 1 at concentrations of between about 25 nM and about 100 nM, or DMSO for 48 hours. The cell suspensions were pelleted, and RNA was extracted with varying concentrations. TRIzol Reagent was used to extract total RNA from SYO1, and HS-SY-II cell pellets, and strands were synthesized using Protoscript II (NEB) with poly A selection. qPCR was performed using TaqMan Gene Expression Master Mix and FAM probes for SUMO2, HPRT, and GAPDH. Paired-end (2×100 bp) sequencing was performed on the NovaSeq 6000 Sequencing System. Data Processing and QC was completed using FastQC and QC summarization was done by MultiQC. Mapping to human genome hg38 v84 was done using STAR sequence aligner, and expression was estimated using RSEM. DESeq2 was used for differential expression analysis, and IPA algorithm was used in evaluation of upstream pathway regulators.

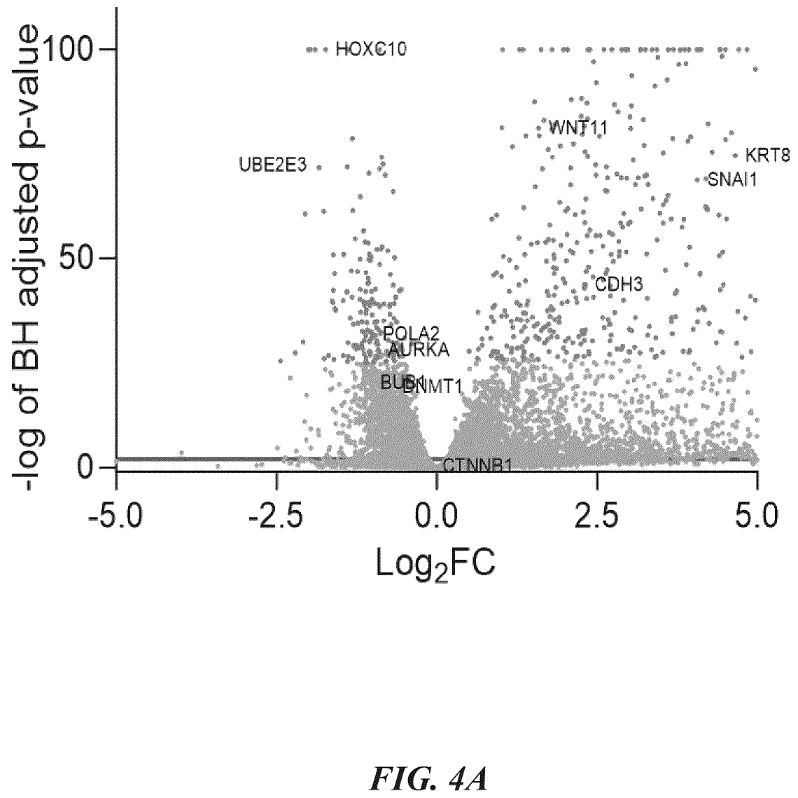

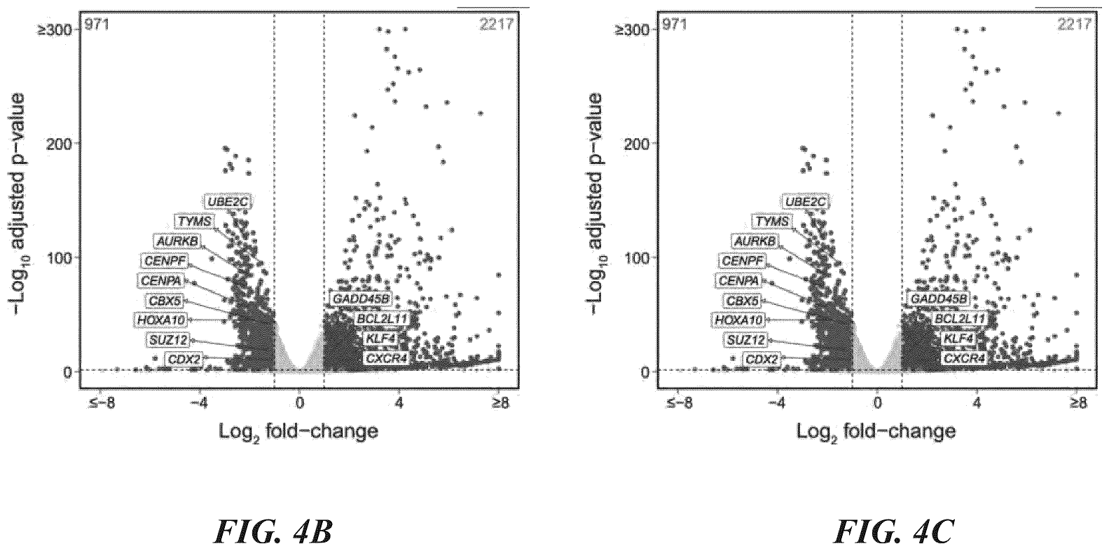

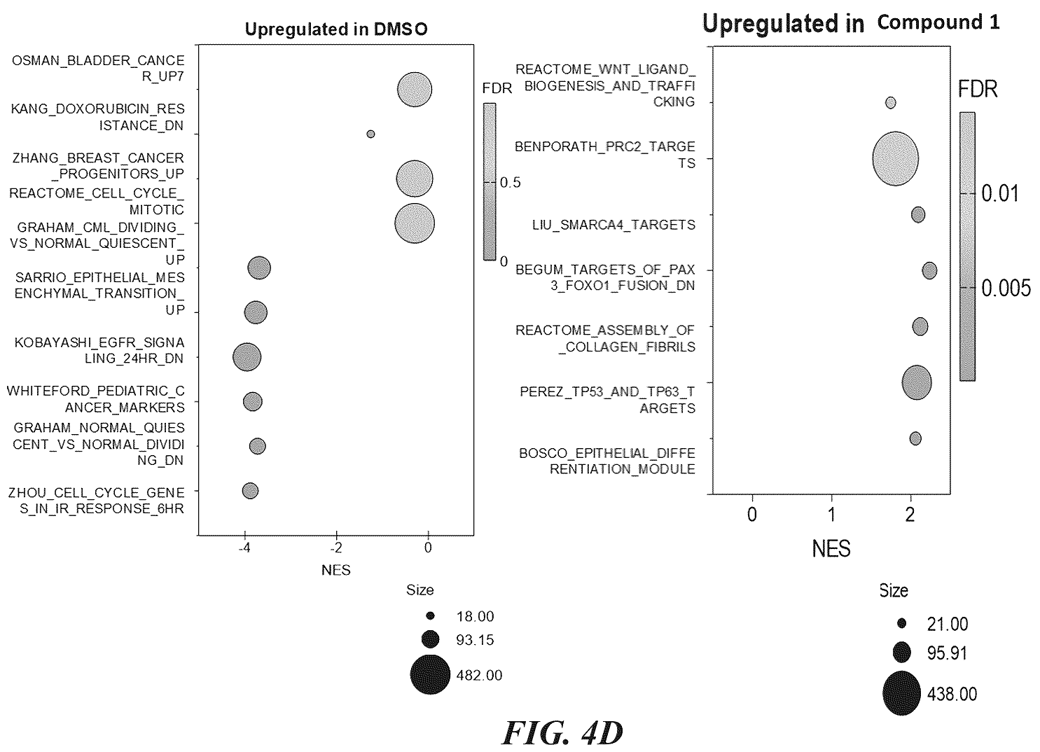

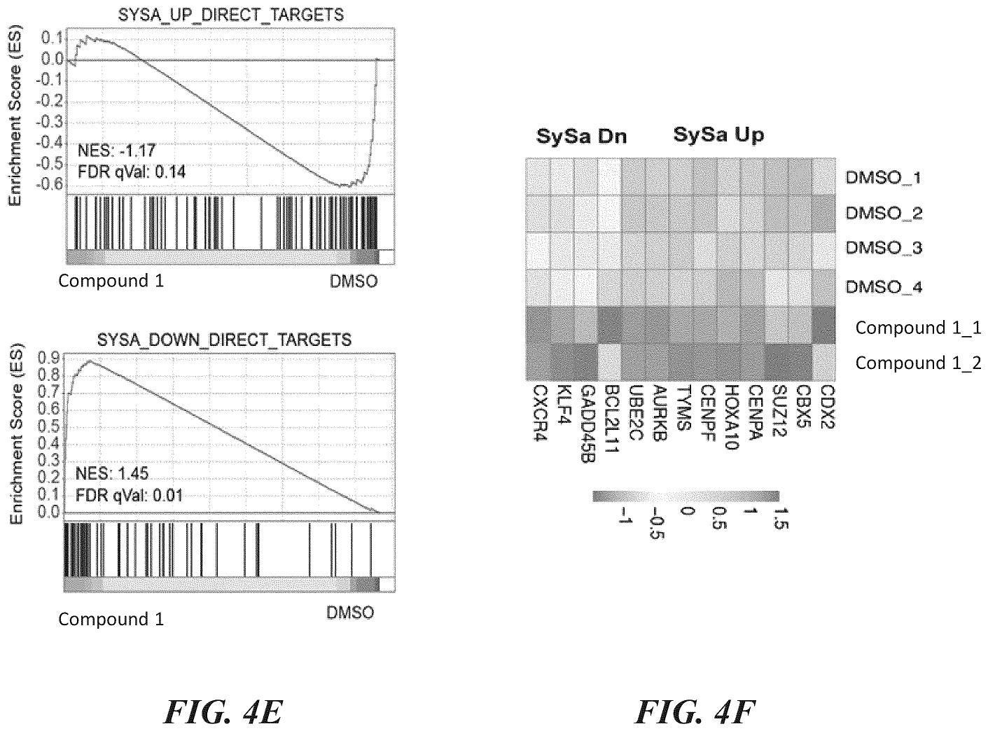

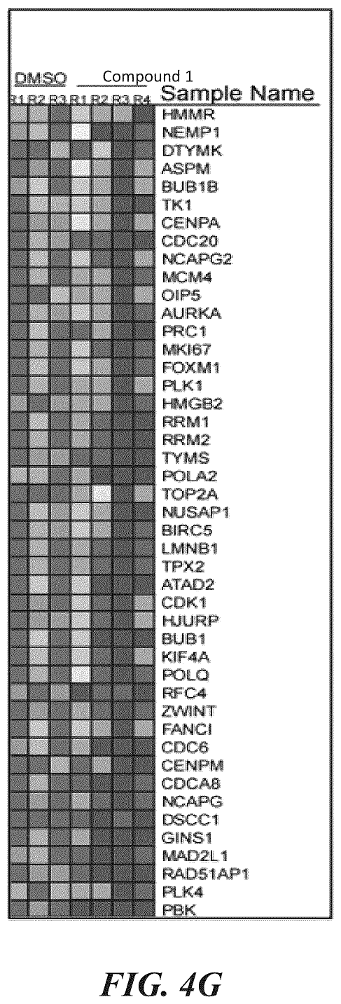

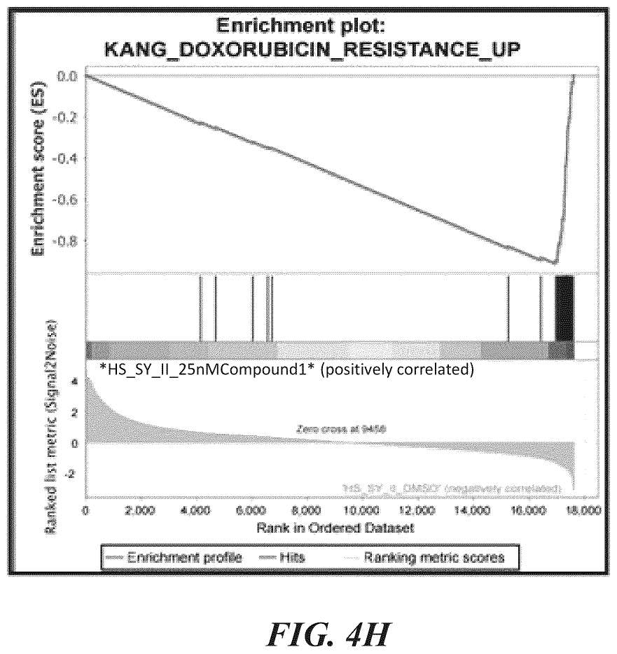

The RNA-seq reads were analyzed for each sample to the human reference genome GRCh38 using STAR aligner. Expression estimation was conducted using RSEM followed by differential expression calling using DESeq2. For target genes of interest in both HS-SY-II and SYO1, a total of 960 differentially expressed target genes of interest (DEGs) were analyzed using sgRNA log fold change values to measure negative log of BH adjusted p-values indicating amount of sgRNA uptake for gene editing (FIG. 4A). Additional screens were performed on HS-SY-II and SYO1 cells, this time with a total of 1100 DEGs detected using the threshold of |fold change|>2 and adjusted p-value<0.01, of which 908 and genes were upregulated or downregulated, respectively. Key cancer-associated genes shown to be upregulated by the SySa fusion29were downregulated by Compound 1 treatment, including CDX2, HOXA10, SUZ12, TYMS, AURKB, (FIG. 4B) and HOXC10 and SMC2 (FIG. 4C)

Upregulation of target genes of interest activated by the chimeric SS18-SSX fusion protein in DMSO media and in media containing Compound 1 were mapped with size and Functional Divergence Ratio (FDR) quantified, based on Nuclear Export Signal (NES) values (FIG. 4D). Expression of various target genes of interest, upregulation, and downregulation by SS18-SSX fusion genomes, were measured in contact with varying concentrations of Compound 1 (FIG. 4E). Heat maps were created mapping the various target genes of interest cultured in media containing only DMSO, compared to media containing Compound 1, for multiple samples (FIG. 4F-4G). Enrichment scores were calculated using gene set enrichment analysis for HS-SY-II cells in contact with DMSO and varying concentrations of Compound 1 for target genes of interest (FIG. 4H). Additional experiments comparing gene expression differences due to DMSO or Compound 1 on SYO-II and HS-SY-II cells were performed. Not only were SS18-SSX-regulated genes affected by Compound 1 treatment, but other genes also impacted included the HOX genes: HOXC6, HOXA10 as well as SRSF1 and TYMS. (FIG. 4I).

SUMO2 Target Gene Knockdown Via shRNA Analysis

shRNAs targeting SUMO2 were cloned into a tetracycline-inducible plasmid and expressed in HS-SYII cells. Small hairpin RNAs (shRNAs) for SUMO2 were cloned into the all-in-one-Tet vector and packaged into lentivirus using pMD and pPax2. A cell suspension containing about 300,000 HS-SY-II and SYO1 cells were seeded into six-well culture plates overnight for about 12-14 hours. Transfections were performed with Lipofectamine 2000 reagent. At about 48 hours after transfection, media was changed and puromycin selected for about 2 days. After selection, cells were subjected to a previously determined amount of doxycycline, about 4.5 μg/ml, for about 48 hours. Cells were harvested for qPCR quantification and western blot analysis.

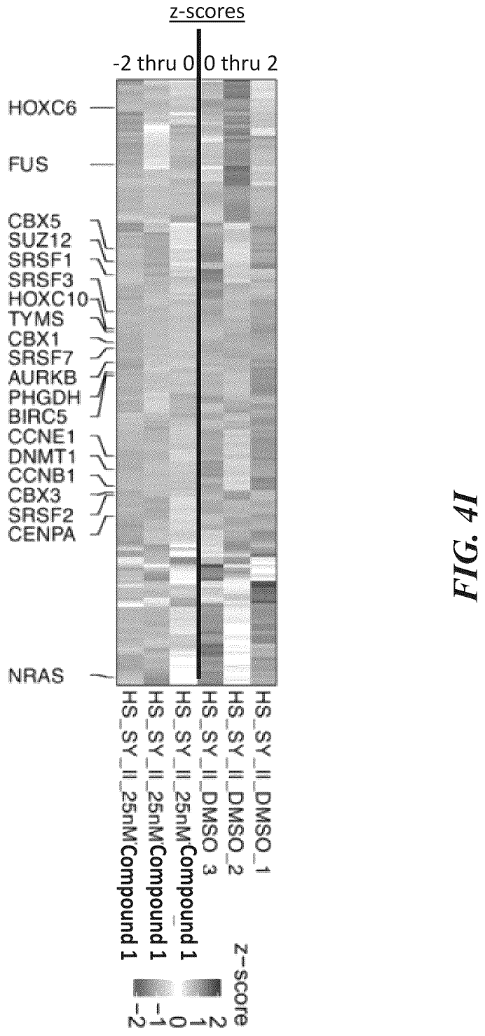

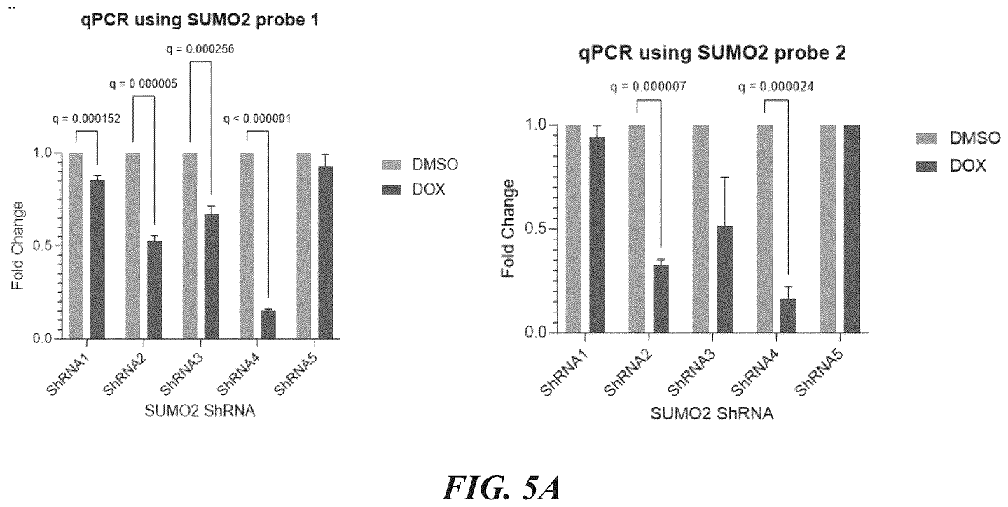

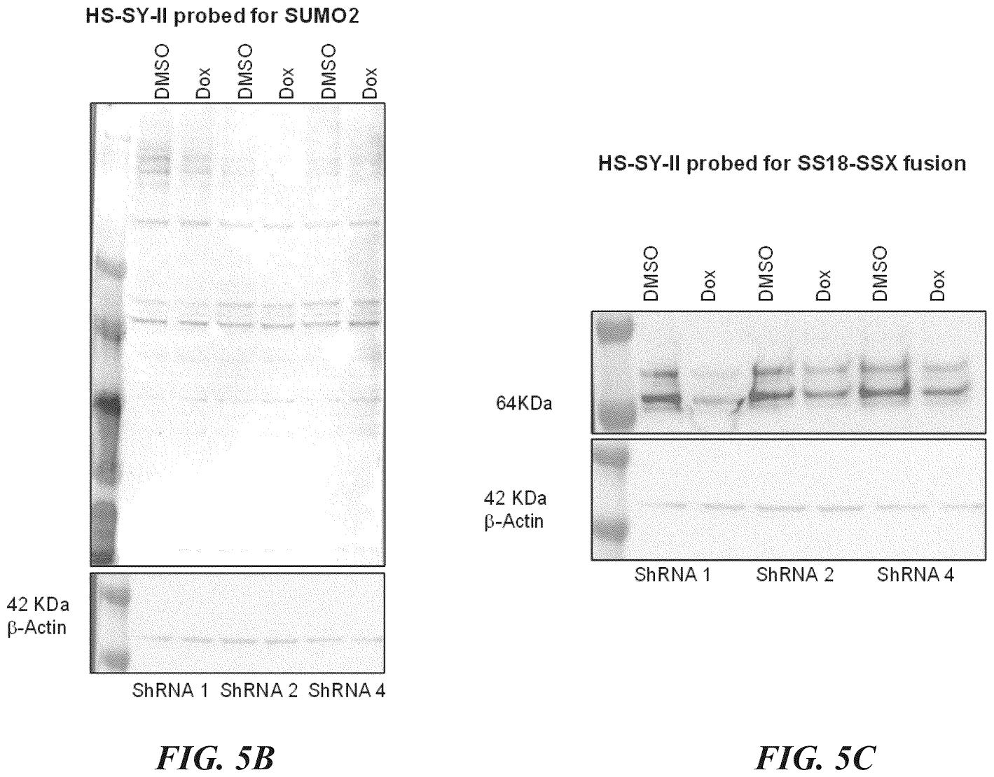

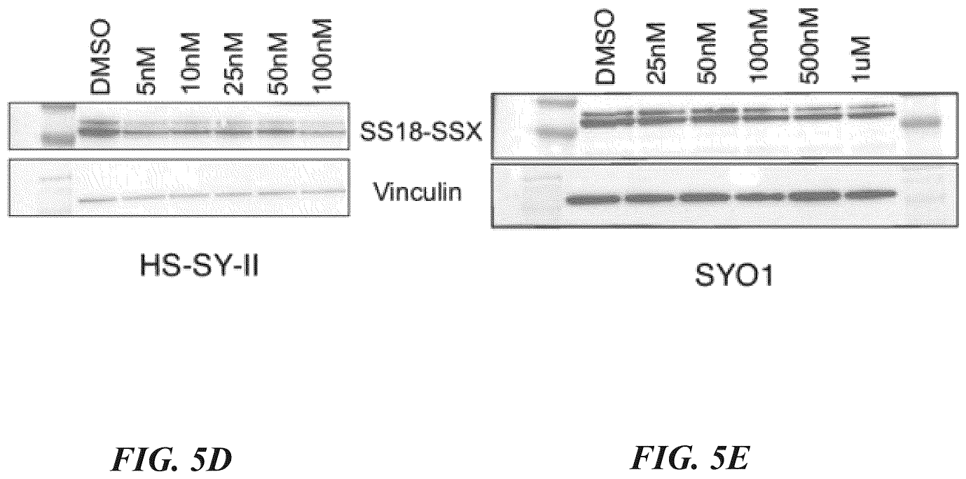

qPCR results were used for validation of knockdown of SUMO2 transcript expression. Multiple qPCR probes were used, and fold change values in SUMO2 shRNA were measured for each probe, including shRNA1, shRNA2, shRNA3, shRNA4, and shRNA5 (FIG. 5A). Western blots were performed on HS-SY-II cells probed for SS18-SSX fusion and for SUMO2 using shRNA probes including shRNA1, shRNA2, and shRNA4 (FIG. 5B-FIG. 5C).

The effect of Compound 1 was compared to the effect of SUMO2 knockdown by shRNA. Compound 1 treatment led to a reduction in the levels of SS18-SSX1 protein in the HS-SY-II cell line (FIG. 5D) and SS18-SSX1 fusion in the SYO1 cell lines. (FIG. 5E) These results provide a striking demonstration that SUMO2 inhibition modulates the levels of oncogenic fusion proteins that drive sarcomagenesis in Synovial Sarcoma.

Example 6: Validation of Genetic Knockdown with Biomarkers

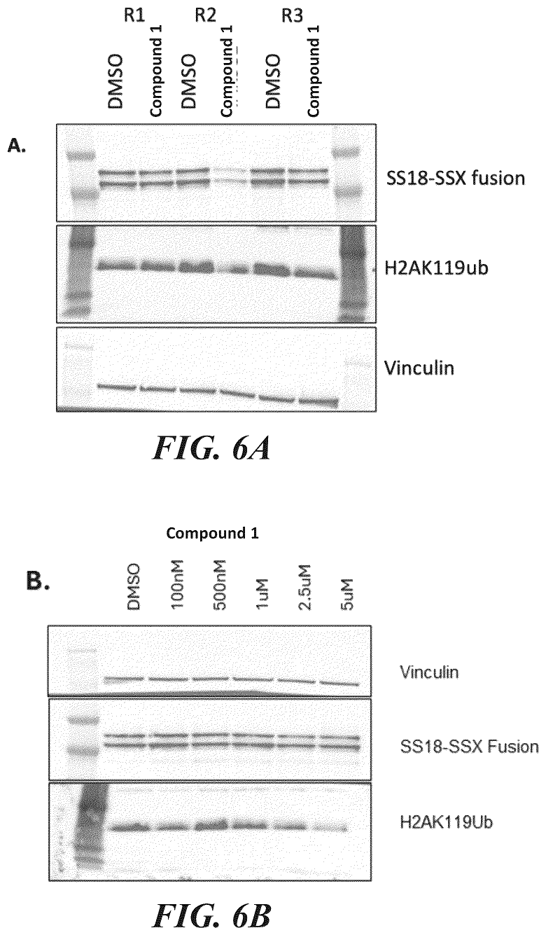

To further validate the knockdown of SUMO2 transcript expression, biomarkers such as H2AK119Ub were investigated as an indication of gene repression. Western blots were performed on SySa cell lines, including line 1273/99 treated with 50 nM of Compound 1 (FIG. 6A). Various probes were used corresponding to the SS18-SSX fusion protein, H2AK119Ub, and Vinculin as a control (FIG. 6A). To determine dose-dependent biomarker differences, western blots were also performed on YAMATO-SS cells (FIG. 6B). The YAMATO-SS cells were treated with various doses of Compound 1, including 100 nM, 500 nM, 1 μM, 2.5 μM, and 5 μM (FIG. 6B). Various probes were used corresponding to the SS18-SSX fusion protein, H2AK119Ub, and Vinculin as a control for each of the dosages of Compound 1 (FIG. 6B).

### Example 7: Compound 1 Causes SS18-SSX1 Eviction from Chromatin In Vitro

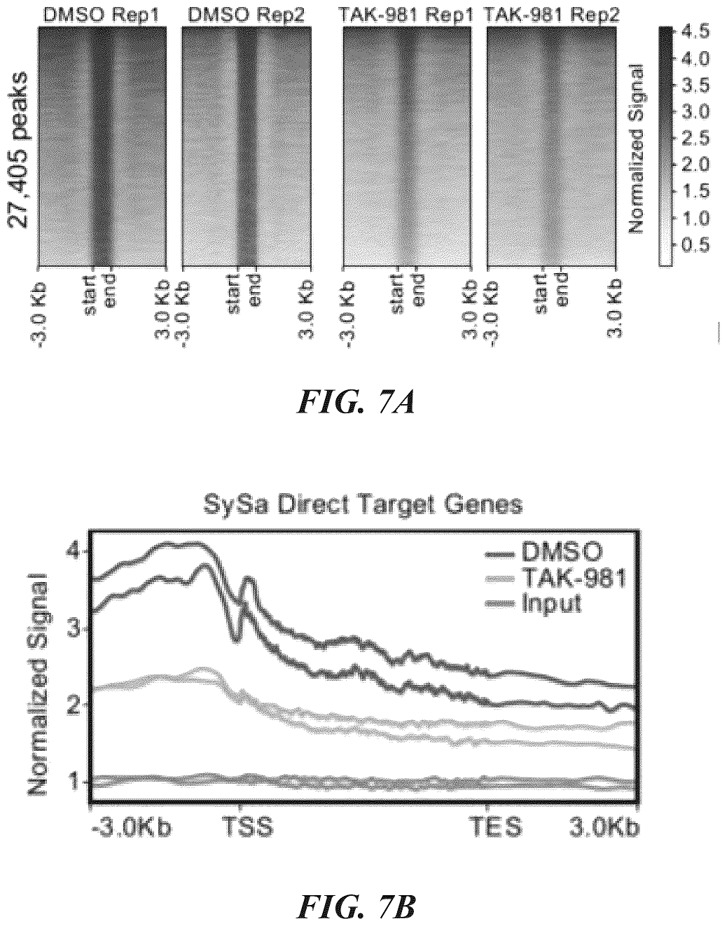

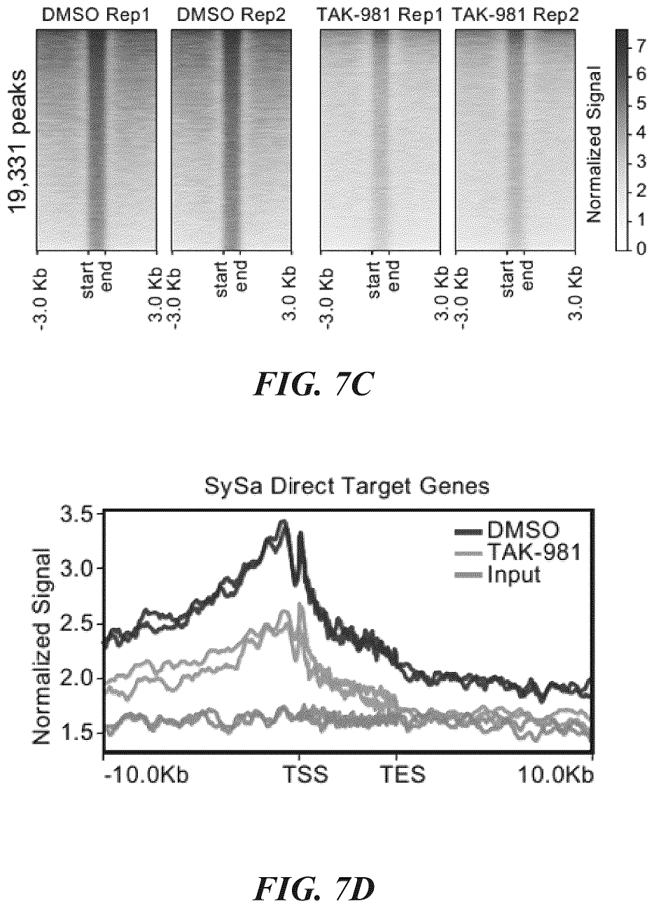

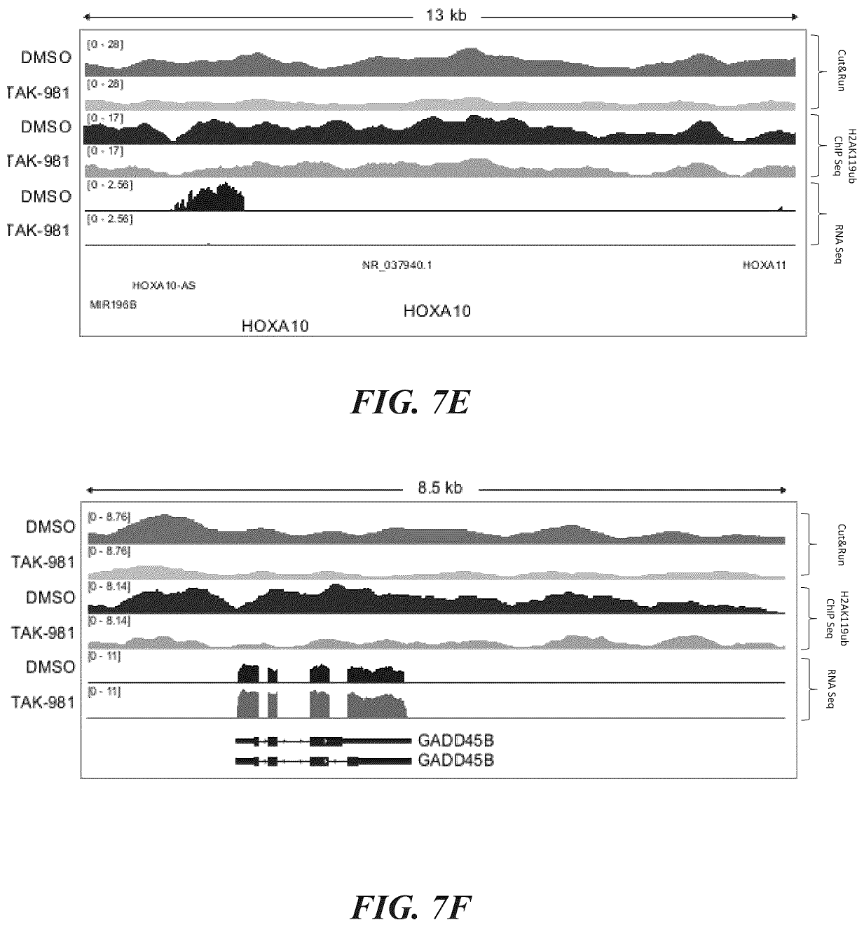

Subsequent studies sought to determine the mechanism behind Compound 1 treatment and the observed attenuation of SS18-SSX expression activity. Initial studies utilizing Cleavage Under Targets & Release Using Nuclease (CUT&RUN) focused on determining whether Compound 1 affects the chromatin occupancy of the SS18-SSX fusion protein. These studies demonstrated a substantial decrease in SS18-SSX2 fusion genomic occupancy as assessed using spike-in normalized CUT&RUN analysis in the Compound 1-treated compared to vehicle-treated arms (FIGS. 7A-B). Specifically, Compound 1 treatment of SYO1 cells showed a 1.87-fold reduction in genome-wide chromatin binding signal of the SS18-SSX fusion compared to the DMSO treated cells, as computed from fraction of reads in peaks (FRiP) measured using consolidated peaks in DMSO replicates. A meta-analysis of the fusion-binding signal at synovial sarcoma target genes revealed a reduction in the fusion binding with the maximum signal centered around the transcription start site. Since increased H2AK119ub deposition has been linked to the pathogenic activity of the SS18-SSX fusions, H2AK119 ubiquitination was assessed in Compound 1-treated cells.

There was a marked reduction in H2AK119ub in SYO1, HS-SY-II, 1273/99 and Aska cell lines treated with Compound 1 as assessed using immunoblotting (FIG. 7C). Furthermore, chromatin immunoprecipitation (ChIP)-sequencing of H2AK119ub showed that like the loss of SSX-SS18 expression, there was a substantial reduction in H2AK119ub in Compound 1 compared to DMSO-treated cells genome-wide (FIG. 7D). Specifically, there was a 1.53-fold reduction of genome-wide H2AK119ub levels in Compound 1 versus DMSO-treated SYO1 cells, computed as fraction of reads in peaks (FRiP) measured using consolidated peaks in DMSO replicates. Genes including the SS18-SSX-activated targets HOXA10 and SOX8 lost SS18-SSX occupancy and showed reduced expression upon Compound 1 treatment (FIG. 7E). Concomitantly, SS18-SSX-repressed targets such as GADD45B showed diminished SS18-SSX fusion occupancy, and reduced H2AK119ub, and showed increased expression (de-repression) following Compound 1 treatment (FIGS. 7F). These results further reinforce the notion that Compound 1 treatment reverses the transcriptional activity of the pathogenic SS18-SSX fusion.

### Example 8: Repeated Dosing of Compound 1 Reduces SySa Flank Tumor Size In Vivo

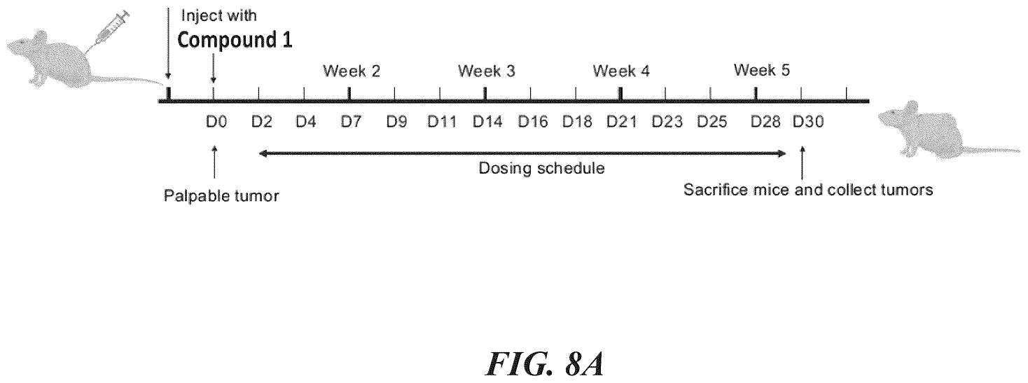

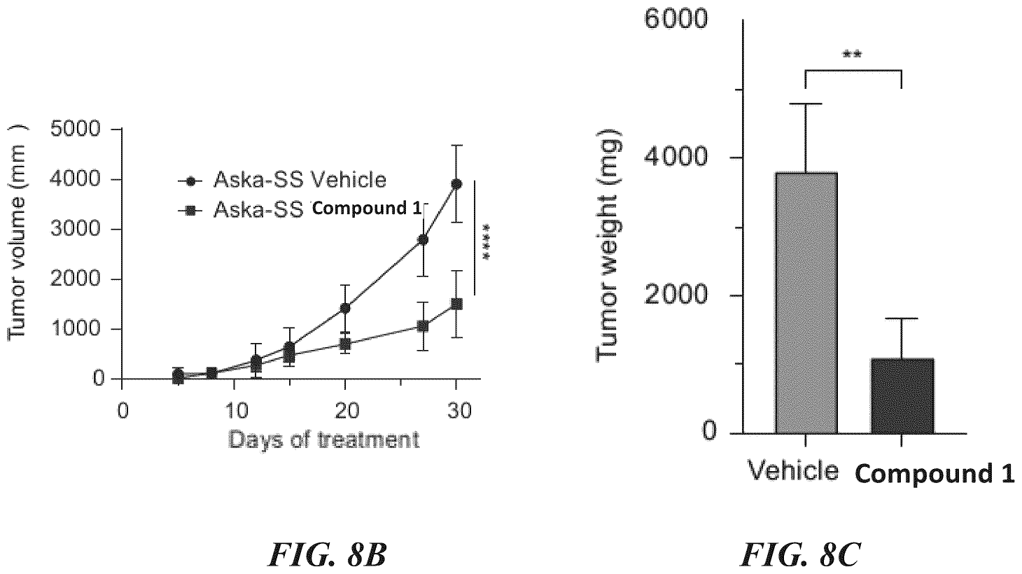

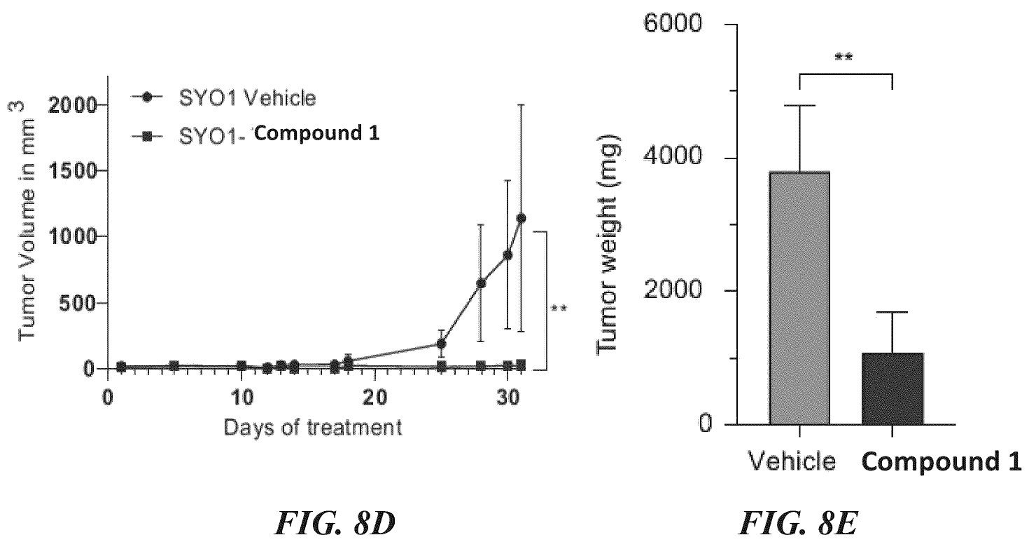

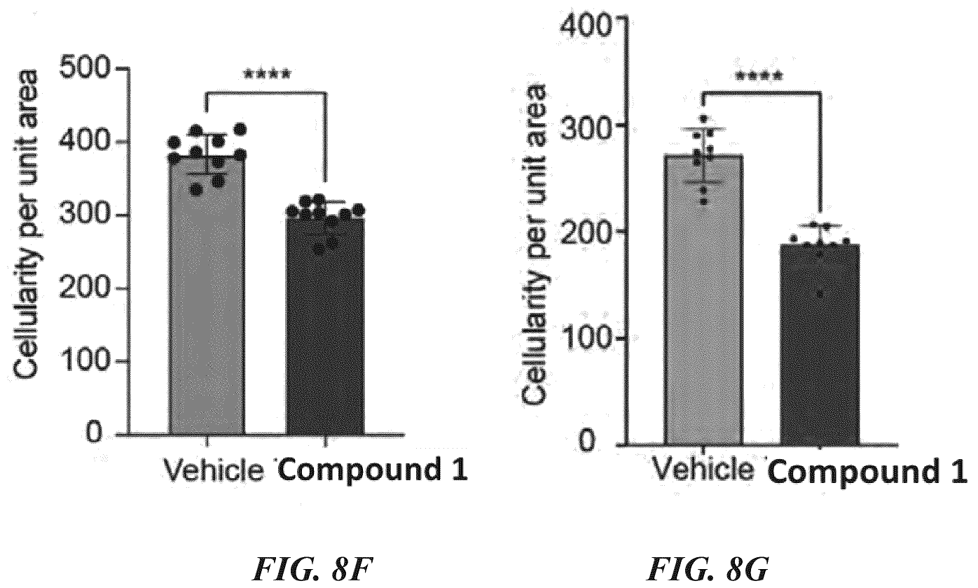

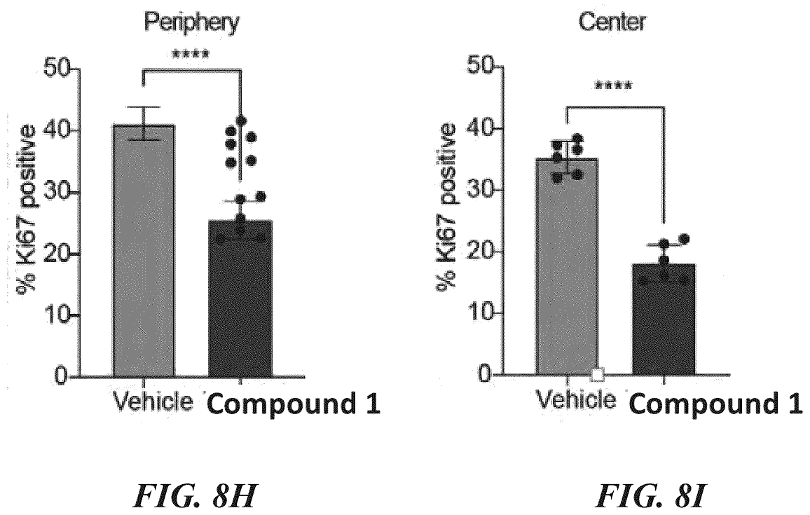

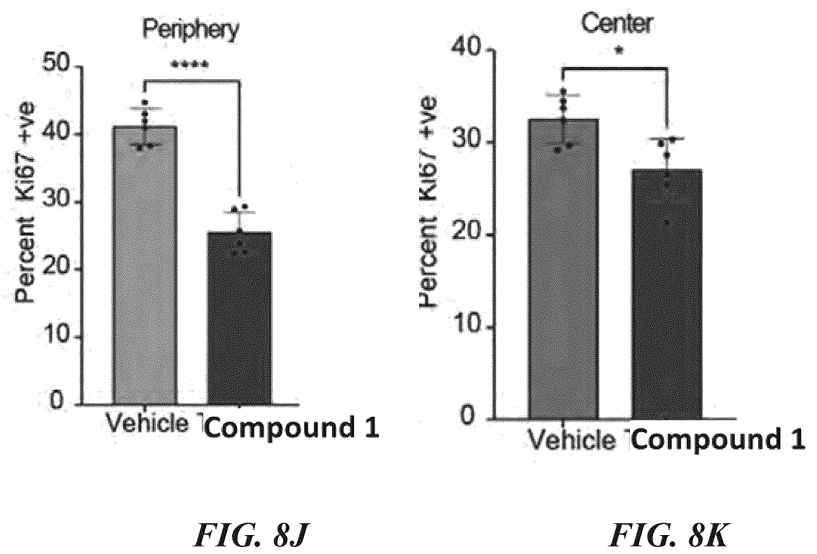

To determine the antitumor activity of Compound 1 in vivo, either Aska-SS cells (harboring the SS18-SSX1 fusion) or SYO1 cells (harboring the SS18-SSX2 fusion) were injected into the flanks of nude mice. When tumors became palpable, mice were treated with 25 mg/kg of Compound 1 or vehicle. A dosing schedule of 3 intraperitoneal injections a week for 5 weeks was maintained (FIG. 8A), Consistent with the in vitro assays, Compound 1-treated mice showed a reduction of tumor growth when compared to vehicle-treated mice. Tumor volumes and weights from Aksa-SS (FIGS. 8B-C) and SYO1 (FIGS. 8D-E) flank tumors were significantly reduced in the Compound 1-treated arm.

Flank tumors were harvested, sectioned, and stained with hematoxylin and eosin and analyzed with IHC for tumoricidal markers. Quantification of tumor sections stained with hematoxylin and eosin showed a marked reduction in the number of cells per unit area within Compound 1-treated Aska-SS (FIG. 8F) and SYO1 (8G) tumors when compared to the vehicle-treated tumors.

Additionally, Ki-67 staining revealed a ˜60% decrease in Ki67 positivity in Aska-SS (FIG. 8H-I) and SYO1 (FIG. 8J-K) tumors treated with Compound 1 in comparison with the vehicle-treated tumors—indicating decreased proliferation. Taken altogether, this data demonstrates that Compound 1 efficiently inhibits growth of SS18-SSX fusion-containing Aska-SS and SYO1 in an animal model.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. Various alternatives to the embodiments of the present disclosure may be employed in practicing the present disclosure. It is intended that the following claims define the scope of the present disclosure and that methods and structures within the scope of these claims and their equivalents be covered thereby.

1. A method of reducing proliferation in a population of synovial sarcoma cells comprising treating the population with a compound inhibiting the conjugation of a small ubiquitin-like modifier 2 (SUMO2), wherein the inhibiting compound is Compound 1, or pharmaceutically acceptable salt thereof, having the structure:

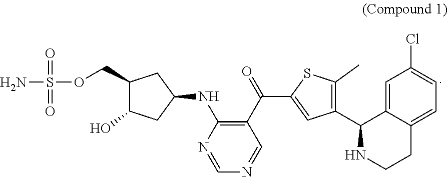 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

2. (canceled)

3. (canceled)

4. (canceled)

5. The method of claim 1, wherein the method induces apoptosis or necrosis, or both, in the population of synovial sarcoma cells.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A method of reducing an expression of a SS18-SSX fusion protein in a population of cancer cells in a subject in need thereof, wherein the method comprises administering to the subject Compound 1, or pharmaceutically acceptable salt thereof, having the structure:

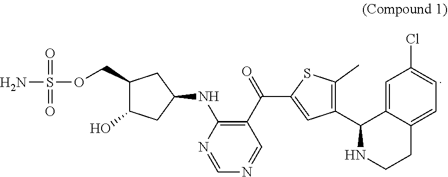 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

26. The method of claim 25, wherein the population of cancer cells comprises one or more sarcoma cells.

27. The method of claim 25, wherein the population of cancer cells comprises one or more synovial sarcoma cells.

28. The method of claim 25, wherein the SS18-SSX fusion protein comprises an SS18 domain and an SSX domain.

29. The method of claim 28, wherein the C-terminus of the SS18 domain is linked to the N-terminus of the SSX domain.

30. The method of claim 25, wherein the SS18 domain comprises the SEQ ID NO:1.

31. The method of claim 25, wherein the SSX domain is selected from the group consisting of an SSX1 domain, an SSX2 domain, an SSX3 domain, and an SSX4 domain.

32. The method of claim 31, wherein an SSX1 domain disclosed herein comprises SEQ ID NO:2, the SSX2 domain comprises SEQ ID NO:3; the SSX3 domain comprises SEQ ID NO:4; or the SSX4 domain comprises SEQ ID NO:5.

33. A method of treating synovial sarcoma comprising administering to a subject in need thereof a Compound 1, or pharmaceutically acceptable salt having the structure:

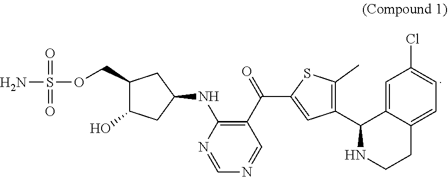 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

34. The method of claim 33, wherein the administered compound dose is within a range of 1 mg to 150 mg.

35. The method of claim 33, wherein the administered compound dose is between 10 mg and 120 mg.

36. The method of claim 33, wherein the administered compound dose is between 1 mg and 75 mg.

37. The method of claim 33, wherein the compound is administered to an adult subject.

38. The method of claim 33, wherein the compound is administered to a pediatric subject.

39-71. (canceled)


SEQUENCE LISTING (5 sequences)

SEQ ID NO: 1 - protein, 418 aa, synthetic construct
MSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGHNDYGYQQPSYPEQGYDRPYEDSSQHYYEGGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 2 - protein, 188 aa, synthetic construct
MNGDDTFAKRPRDDAKASEKRSKAFDDIATYFSKKEWKKMKYSEKISYVYMKRNYKAMTKLGFKVTLPPFMCNKQATDFQGNDFDNDHNRRIQVEHPQMTFGRLHRIIPKIMPKKPAEDENDSKGVSEASGPQNDGKQLHPPGKANISEKINKRSGPKRGKHAWTHRLRERKQLVIYEEISDPEEDDE

SEQ ID NO: 3 - protein, 188 aa, synthetic construct
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLRERKQLVIYEEISDPEEDDE

SEQ ID NO: 4 - protein, 188 aa, synthetic construct
MNGDDTFARRPTVGAQIPEKIQKAFDDIAKYFSKEEWEKMKVSEKIVYVYMKRKYEAMTKLGFKAILPSFMRNKRVTDFQGNDFDNDPNRGNQVQRPQMTFGRLQGIFPKIMPKKPAEEGNVSKEVPEASGPQNDGKQLCPPGKPTTSEKINMISGPKRGEHAWTHRLRERKQLVIYEEISDPEEDDE

SEQ ID NO: 5 - protein, 188 aa, synthetic construct
MNGDDAFARRPRDDAQISEKLRKAFDDIAKYFSKKEWEKMKSSEKIVYVYMKLNYEVMTKLGFKVTLPPFMRSKRAADFHGNDFGNDRNHRNQVERPQMTFGSLQRIFPKIMPKKPAEEENGLKEVPEASGPQNDGKQLCPPGNPSTLEKINKTSGPKRGKHAWTHRLRERKQLVVYEEISDPEEDDE